(19) United States
(12) Patent Application Publication
(10) Pub. No.: US 2022/0411678 A1
(43) Pub. Date: Dec. 29, 2022

(54) REVERSIBLE ADHESIVES

(71) Applicant: Alfred E. Mann Institute for Biomedical Engineering at the University of Southern California, Los Angeles, CA (US)

(72) Inventors: Jenna Lubet, Los Angeles, CA (US); Nan Chen, Los Angeles, CA (US); Nichole Escamilla, Los Angeles, CA (US); Cesar Blanco, Los Angeles, CA (US); Mark Edward Thompson, Los Angeles, CA (US)

(21) Appl. No.: 17/755,212

(22) PCT Filed: Oct. 22, 2020
(86) PCT No.: PCT/US2020/056910

§ 371 Date: Apr. 22, 2022

Related U.S. Application Data
(60) Provisional application No. 62/926,290, filed on Oct. 25, 2019.

(51) Int. Cl. C09J 133/02; C09J 133/26; C08F 220/06; C08F 220/56; C08F 222/10; A61L 15/24; A61L 15/58

(52) U.S. Cl. CPC C09J 133/02; C09J 133/26; C08F 220/06; C08F 220/56; C08F 222/102; A61L 15/24; A61L 15/58; C09J 2433/00

(57) ABSTRACT

Adhesives, particularly reversible adhesives, reversible adhesive hydrogel meshes and polymer formulations that may be used in preparation of the reversible adhesive hydrogel meshes are disclosed. The polymer formulations may comprise a reversible monomer of a reversible adhesive polymer, acrylic acid (AA), an acrylate cross-linker, a photo-initiator for free radical polymerization, and a solvent. The disclosure also relates to a wound dressing comprising the reversible adhesive hydrogel meshes. Such wound dressings are particularly suitable for treatment of damaged sensitive tissue, for example, wounds formed on a fragile skin.

### CROSS-REFERENCE TO RELATED APPLICATION

This application claims the benefit of U.S. provisional patent application 62/926,290, entitled “Reversible Adhesives,” filed Oct. 25, 2019, attorney docket number AMISC.011PR. The entire content of this application is incorporated herein by reference.

### BACKGROUND

### Technical Field

This disclosure relates generally to adhesives and particularly to reversible adhesives. This disclosure also relates to reversible adhesive hydrogel meshes. This disclosure also relates to polymer formulations that may be used in preparation of the reversible adhesive hydrogel meshes. This disclosure also relates to a wound dressing comprising the reversible adhesive hydrogel meshes. Such wound dressings are particularly suitable for treatment of damaged sensitive tissue, for example, wounds formed on a fragile skin.

### Description of Related Art

Wound dressings incorporating pressure sensitive adhesives are well known and commercially available. Examples of wound dressings are adhesive bandages, transdermal drug patches and surgical patches.

Although such adhesives immediately adhere to a substrate when pressure is applied, their removal from the substrate becomes a hurdle later. For example, a bandage manufactured by using a pressure sensitive adhesive can easily be applied to a wound formed on a skin with high adherence. However, when this bandage is desired to be removed from the skin to replace it with another bandage or after completion of treatment of the wound, a force needs to be applied to counteract high adherence of the bandage, which may cause pain to the patient and/or damage to the wound or the healthy tissue surrounding the wound. Such hurdles are very frequently encountered during interventions to wounds by trained personnel at medical institutions as well as individuals at home.

### SUMMARY

This disclosure relates to adhesives and particularly to reversible adhesives. This disclosure also relates to reversible adhesive hydrogel meshes. This disclosure also relates to polymer formulations that may be used in preparation of the reversible adhesive hydrogel meshes. The polymer formulations may comprise a reversible monomer of a reversible adhesive polymer, acrylic acid (AA), an acrylate cross-linker, a photo-initiator for free radical polymerization, and a solvent. This disclosure also relates to a wound dressing comprising the reversible adhesive hydrogel meshes. Such wound dressings are particularly suitable for treatment of damaged sensitive tissue, for example, wounds formed on a fragile skin.

In this disclosure, the reversible adhesive hydrogel mesh may be prepared by reacting a polymer formulation. The polymer formulation may include a reversible monomer of a reversible adhesive polymer, acrylic acid (AA), an acrylate cross-linker, a photo-initiator for free radical polymerization, and a solvent. In one example, the polymer formulation may include N-isopropylacrylamide (NiPAM), acrylic acid (AA), a covalent diacrylate cross-linker or a covalent triacrylate cross-linker, a photo-initiator for free radical polymerization, and dimethylsulfoxide.

The reversible monomer of a reversible adhesive polymer may comprise N-methyl-N-n-propylacrylamide, N-n-propylacrylamide, N-methyl-N-isopropylacrylamide, N-n-propylmethacrylamide, N-isopropylacrylamide, N-n-diethylacrylamide, N-isopropylmethacrylamide, N-cyclopropylacrylamide, N-ethylmethyacrylamide, N-methyl-N-ethylacrylamide, N-cyclopropylmethacrylamide, N-ethylacrylamide, N,N-diethylacrylamide, or a mixture thereof.

The acrylate cross-linker may include a multifunctional acrylate cross-linker. Examples of the multifunctional acrylate cross-linker may be a bifunctional acrylate cross-linker, a trifunctional acrylate cross-linker, a tetrafunctional acrylate cross-linker, a hexafunctional acrylate cross-linker, or a mixture thereof. Examples of the acrylate cross-linker may be N,N′-methylene bisacrylamide (BIS), a poly(ethylene glycol) diacrylate (PEG-DA), trimethylolpropane triacrylate (TMP-TA), a trimethylolpropane ethoxylate triacrylate, (TMPE-TA), or a mixture thereof. Examples of the acrylate cross-linker may be a covalent diacrylate cross-linker, and wherein the covalent diacrylate cross-linker may be N,N-methylene bisacrylamide (BIS), poly(ethylene glycol) diacrylate (PEG-DA), poly(propylene glycol) diacrylate (PPG-DA), or a mixture thereof. Examples of the poly(ethylene glycol) diacrylate (PEG-DA) may be PEG-DA with an average molecular weight, Mn of 250 kDa; PEG-DA with an average molecular weight, Mn of 575 kDa; PEG-DA with an average molecular weight, Mn of 700 kDa, or a mixture thereof. An example of PPG-DA may be PPG-DA with an average molecular weight Mn of 800 kDa. Examples of the trimethylolpropane ethoxylate triacrylate, (TMPE-TA) may be TMPE-TA with an average molecular weight, Mn of 428 kDa; TMPE-TA with an average molecular weight, Mn of 912 kDa, or a mixture thereof. An example of the acrylate cross-linker may be trimethylolpropane triacrylate.

The reversible adhesive hydrogel mesh may not have a definable molecular weight because it is covalently cross-linked across and indefinite volume area.

In this disclosure, the polymer formulation may further include an acrylate co-monomer. Examples of the acrylate co-monomer may include 2-ethylhexyl acrylate, di(ethylene glycol) 2-ethylhexyl acrylate, poly(ethylene glycol) methyletheracrylate (PEG-MEA), poly(propylene glycol) acrylate (PPG-A), sodium acrylate, 2-hydroxyethyl acrylate, 2-hydroxymethylethyl acrylate, dopamine acrylate, or a mixture thereof.

In this disclosure, the polymer formulation may further include a vinyl-functionalized co-monomer. Examples of the vinyl-functionalized co-monomer may include vinyl acetate, 1-vinyl-2-pyrrolidinone, N-vinylcaprolactam, ethyl vinyl ether, isobutyl vinyl ether, propyl vinyl ether, butyl vinyl ether, cyclohexyl vinyl ether, tert-butyl vinyl ether, dodecylvinyl ether, 2-ethylhexyl vinyl ether, isopropyl vinyl ether, isobutyl vinyl ether, octyl vinyl ether, ethylene glycol vinyl ether, diethyl vinyl orthoformate, di(ethylene glycol) vinyl ether, 1,4-butanediol vinyl ether, 2-chloroethyl vinyl ether, or a mixture thereof.

In this disclosure, the polymer formulation may further include an additive. Examples of the additive may be laponite, sodium polyacrylate, sodium alginate, tannic acid, chitosan, a silver particle, an aloe extract, propanediol, 1,4-butanediol, 1,5-pentanediol, hexanediol, octanediol, or a mixture thereof.

In this disclosure, the solvent may include dimethylformamide (DMF), water, ethanol, ethyl acetate, propylene carbonate, dimethyl sulfoxide (DMSO), propylene glycol (PPG), or a mixture thereof. In another example, the solvent my include dimethyl sulfoxide (DMSO).

In this disclosure, a lower critical solution temperature (LCST) of the reversible adhesive hydrogel mesh may be in a range of 15° C. to 35° C.

In this disclosure, a concentration of the reversible monomer of a reversible adhesive polymer in the polymer formulation may be in a range of 1 w/v % to 20 w/v %. In another example, concentration of the reversible monomer of a reversible adhesive polymer in the polymer formulation may be in a range of 5 w/v % to 15 w/v %.

In this disclosure, a concentration of acrylic acid in the polymer formulation may be in a range of 1 w/v % to 20 w/v %. In another example, a concentration of acrylic acid in the polymer formulation may be in a range of 5 w/v % to 15 w/v %.

In this disclosure, a concentration of the acrylate cross linker in the polymer formulation may be in a range of 0.1 w/v % to 5 w/v %. In another example, a concentration of the acrylate cross linker in the polymer formulation may be in a range of 1 w/v % to 2 w/v %.

In this disclosure, the catalyst may comprise potassium persulfate, azobisisobutyronitrile, 4,4′-azobis(4-cyanovaleric acid), 1,1′-azobis(cyclohexanecarbonitrile), or a mixture thereof.

In this disclosure, the catalysts may be a photo-initiator. The photo-initiator may comprise 2-hydroxy-4′-(2-hydroxyethoxy)-2-methylpropiophenone.

In this disclosure, the concentration of the catalyst (e.g. photo-initiator) may be in a range of 0.001 w/v % to 0.5 w/v %. Or, the concentration of the catalyst (e.g. photo-initiator) may be in a range of 0.001 w/v % to 0.2 w/v %.

In this disclosure, adhesive strength of the reversible adhesive hydrogel mesh at a first temperature may be higher than that of the reversible adhesive hydrogel mesh at a second temperature; and wherein the first temperature is higher than the second temperature.

In this disclosure, adhesive strength of the reversible adhesive hydrogel mesh at the first temperature may be in a range of 0.70 N/cm² to 2.20 N/cm², or 0.70 N/cm² to 0.90 N/cm²; when the adhesive strength of the reversible adhesive hydrogel mesh is characterized using ASTM method F2258-05.

In this disclosure, adhesive strength of the reversible adhesive hydrogel mesh at the second temperature may be in a range of 0.40 N/cm² to 0.90 N/cm², or 0.50 N/cm² to 0.80 N/cm², or 0.53 N/cm² to 0.70 N/cm²; when the adhesive strength of the reversible adhesive hydrogel mesh is characterized using ASTM method F2258-05.

In this disclosure, adhesive strength of the reversible adhesive hydrogel mesh at the first temperature is in a range of 0.70 N/cm² to 2.20 N/cm², or 0.70 N/cm² to 0.90 N/cm²; and wherein the adhesive strength of the reversible adhesive hydrogel mesh at the second temperature is in a range of 0.40 N/cm² to 0.90 N/cm², or 0.50 N/cm² to 0.80 N/cm², or 0.53 N/cm² to 0.70 N/cm²; when the adhesive strength of the reversible adhesive hydrogel mesh is characterized using ASTM method F2258-05.

In this disclosure, the reversible adhesive hydrogel mesh may be a reversible adhesive hydrogel mesh that adheres to a (human) tissue. The tissue may comprise any tissue. For example, the tissue may comprise epithelial tissue (epithelium), connective tissue, muscle tissue, nervous tissue, or a composite thereof. For example, the tissue may comprise an epithelial tissue. The epithelial tissue may, for example, be a tissue that lines outer surfaces of organs, blood vessels, and inner surfaces of cavities of internal organs throughout a (human) body. An example of the tissue may be a skin. The skin may comprise epidermis, basement membrane, dermis, subcutaneous tissue, or a composite thereof. The skin may be a fragile skin. The fragile skin may be a fragile skin of a human belonging to a pediatric population, a geriatric population, or a human with an injury or disease. The human with an injury or disease may be afflicted with a chronic wound (e.g. a ulcer), a burn injury, or a combination of any of these afflictions.

This disclosure also relates to a wound dressing. This wound dressing may include any reversible adhesive hydrogel mesh of this disclosure. The wound dressing may further include a backing material.

This disclosure also relates to a wound dressing. This wound dressing may include any reversible adhesive hydrogel mesh of this disclosure. The wound dressing may further include an adhesive primer layer between the reversible adhesive hydrogel mesh and a backing material.

This disclosure also relates to a wound dressing. This wound dressing may include any reversible adhesive hydrogel mesh of this disclosure. The wound dressing may further include a release liner material.

This disclosure also relates to a method of preparation of a reversible adhesive hydrogel mesh. This method may include preparing a reaction solution comprising any polymer formulation of this disclosure, reacting the polymer formulation to prepare the reversible adhesive hydrogel mesh, and treating the prepared reversible adhesive hydrogel mesh with a humectant. Examples of the humectant may include glycerol, ethylene glycol, propylene glycol, hexylene glycol, an aloe extract, hyaluronic acid, 2,3-butanediol, butyl ethyl propanediol, or a mixture thereof. In one example, the method of the treating the prepared reversible adhesive hydrogel mesh with a humectant may include washing the reversible adhesive hydrogel mesh with a hydrogel wash solvent (“a first washing”), washing the reversible adhesive hydrogel mesh with a mixture comprising a hydrogel wash solvent and a humectant (“a second washing”) after the first washing, washing the reversible adhesive hydrogel mesh with a mixture comprising a hydrogel wash solvent, a humectant, and water (“a third washing”) after the second washing, and washing the reversible adhesive hydrogel mesh with a mixture comprising a humectant and water (“a fourth washing”) after the third washing, and thereby obtaining a reversible adhesive hydrogel mesh with improved tack. In the fourth wash, the humectant concentration may be equal to or higher than 25 volume percent, equal to or higher than 50 volume percent, or equal to or higher than 75 volume percent in the mixture comprising a humectant and water. In one example, the method of treating the prepared reversible adhesive hydrogel mesh with a humectant may include washing the reversible adhesive hydrogel mesh a mixture comprising humectant and water (“a first washing”) and washing the reversible adhesive hydrogel mesh with a mixture comprising humectant and water (“a second washing”). In the first and second wash, the humectant concentration may be equal to or higher than 25 volume percent, equal to or higher than 50 volume percent, or equal to or higher than 75 volume percent in the mixture comprising a humectant and water.

Another method of preparation of a reversible adhesive hydrogel mesh method may include preparing a reaction solution comprising a polymer formulation of any of the preceding or a following claims, reacting the polymer formulation to prepare the reversible adhesive hydrogel mesh, and treating the prepared reversible adhesive hydrogel mesh with a treatment solution comprising a humectant, and thereby obtaining a reversible adhesive hydrogel mesh with improved tack.

In this disclosure, the treating the prepared reversible adhesive hydrogel mesh may include treating the reversible adhesive hydrogel mesh with a first treatment solution before treating the prepared reversible adhesive hydrogel mesh with a treatment solution comprising a humectant, wherein the first treatment solution may include water and an alcohol; and then treating the reversible adhesive hydrogel mesh with a second treatment solution, wherein the second treatment solution may include a humectant and water.

In this disclosure, the treating the prepared reversible adhesive hydrogel mesh may also include treating the reversible adhesive hydrogel mesh with a treatment solution, wherein the treatment solution comprises a humectant, an alcohol and water; and thereby obtaining a reversible adhesive hydrogel mesh with improved tack.

In this disclosure, the treating the prepared reversible adhesive hydrogel mesh may include treating the reversible adhesive hydrogel mesh with a treatment solution, wherein the treatment solution may comprise a humectant and water; and thereby obtaining a reversible adhesive hydrogel mesh with improved tack.

In this disclosure, the humectant may include glycerol, ethylene glycol, propylene glycol, hexylene glycol, an aloe extract, hyaluronic acid, 2,3-butanediol, butyl ethyl propanediol, or a mixture thereof.

Any combination of above or below exemplary reversible adhesive hydrogel meshes, exemplary chemicals or formulations used in preparation of these meshes, and exemplary methods used in preparation of these meshes are within the scope of this disclosure.

In this disclosure, the humectant concentration of a treatment solution may be equal to or higher than 25 volume percent, equal to or higher than 50 volume percent, or equal to or higher than 75 volume percent in the mixture comprising a humectant and water

These, as well as other components, steps, features, objects, benefits, and advantages, will now become clear from a review of the following detailed description of illustrative examples, the accompanying drawings, and the claims.

Some embodiments relate to a reversible adhesive hydrogel mesh, including cross-linked components of the following monomers:

a reversible monomer of a reversible adhesive polymer, acrylic acid (AA), and an acrylate cross-linker.

In some examples, the reversible monomer of the reversible adhesive polymer includes N-methyl-N-n-propylacrylamide, N-n-propylacrylamide, N-methyl-N-isopropylacrylamide, N-n-propylmethacrylamide, N-isopropylacrylamide (NIPAM), N-n-diethylacrylamide, N-isopropylmethacrylamide, N-cyclopropylacrylamide, N-ethylmethyacrylamide, N-methyl-N-ethylacrylamide, N-cyclopropylmethacrylamide, N-ethylacrylamide, N-n-diethylacrylamide, or a mixture thereof.

In some examples, the acrylate cross-linker includes a multifunctional acrylate cross-linker.

In some examples, the acrylate cross-linker includes a bifunctional acrylate cross-linker, a trifunctional acrylate cross-linker, a tetrafunctional acrylate cross-linker, a hexafunctional acrylate cross-linker, or a mixture thereof.

In some examples, the acrylate cross-linker includes a bifunctional acrylate cross-linker, a trifunctional acrylate cross-linker, or a mixture thereof.

In some examples, the acrylate cross-linker includes N,N-methylene bisacrylamide (BIS), a poly(ethylene glycol) diacrylate (PEG-DA), trimethylolpropane triacrylate (TMP-TA), a trimethylolpropane ethoxylate triacrylate, (TMPE-TA), poly(propylene glycol) diacrylate (PPG-DA), trimethylolpropane triacrylate, or a mixture thereof.

In some examples, the acrylate cross-linker includes a covalent diacrylate cross-linker, and wherein the covalent diacrylate cross-linker comprises N,N-methylene bisacrylamide (BIS), poly(ethylene glycol) diacrylate (PEG-DA), poly(propylene glycol) diacrylate (PPG-DA), or a mixture thereof.

In some examples, the acrylate cross-linker includes PEG-DA with an average molecular weight, Mn of about 250 kDa; PEG-DA with an average molecular weight, Mn of about 575 kDa; PEG-DA with an average molecular weight, Mn of about 700 kDa; PPG-DA with an average molecular weight Mn of about 800 kDa; TMPE-TA with an average molecular weight, Mn of about 428 kDa; TMPE-TA with an average molecular weight, Mn of about 912 kDa; or a mixture thereof.

Some embodiments relate to a reversible adhesive hydrogel mesh prepared by reacting a polymer formulation, wherein the polymer formulation comprises:

a reversible monomer of a reversible adhesive polymer,

acrylic acid (AA),

an acrylate cross-linker,

a catalyst, and

a solvent.

In some examples, the polymer formulation further includes an acrylate co-monomer.

In some examples, the solvent includes dimethylformamide (DMF), water, ethanol, ethyl acetate, propylene carbonate, dimethyl sulfoxide (DMSO), propylene glycol, or a mixture thereof.

In some examples, the catalyst includes a photo-initiator for free radical polymerization.

In some examples, the catalyst includes potassium persulfate, azobisisobutyronitrile, 4,4′-azobis(4-cyanovaleric acid), 1,1′-azobis(cyclohexanecarbonitrile), 2-hydroxy-4′-(2-hydroxyethoxy)-2-methylpropiophenone, or a mixture thereof.

In some examples, the polymer formulation further includes laponite, sodium polyacrylate, sodium alginate, tannic acid, chitosan, a silver particle, an aloe extract, propanediol, 1,4-butanediol, 1,5-pentanediol, hexanediol, octanediol, or a mixture thereof.

In some examples, the polymer formulation further includes an acrylate co-monomer; wherein the acrylate co-monomer comprises 2-ethylhexyl acrylate, di(ethylene glycol) 2-ethylhexyl acrylate, poly(ethylene glycol) methyletheracrylate (PEG-MEA), poly(propylene glycol) acrylate (PPG-A), sodium acrylate, 2-hydroxyethyl acrylate, 2-hydroxymethylethyl acrylate, dopamine acrylate, or a mixture thereof.

In some examples, the polymer formulation further includes a vinyl functionalized co-monomer.

In some examples, the polymer formulation further includes vinyl acetate, 1-vinyl-2-pyrrolidinone, N-vinylcaprolactam, ethyl vinyl ether, isobutyl vinyl ether, propyl vinyl ether, butyl vinyl ether, cyclohexyl vinyl ether, tert-butyl vinyl ether, dodecylvinyl ether, 2-ethylhexyl vinyl ether, isopropyl vinyl ether, isobutyl vinyl ether, octyl vinyl ether, ethylene glycol vinyl ether, diethyl vinyl orthoformate, di(ethylene glycol) vinyl ether, 1,4-butanediol vinyl ether, 2-chloroethyl vinyl ether, or a mixture thereof.

In some examples, a concentration of the reversible monomer in the polymer formulation is in a range of 1 w/v % to 20 w/v %.

In some examples, a concentration of the reversible monomer in the polymer formulation is in a range of 5 w/v % to 15 w/v %.

In some examples, a concentration of acrylic acid (AA) in the polymer formulation is in a range of 1 w/v % to 20 w/v %.

In some examples, a concentration of acrylic acid (AA) in the polymer formulation is in a range of 5 w/v % to 15 w/v %.

In some examples, a concentration of the acrylate cross-linker in the polymer formulation is in a range of 0.1 w/v % to 5 w/v %.

In some examples, a concentration of the acrylate cross-linker in the polymer formulation is in a range of 1 w/v % to 2 w/v %.

In some examples, a concentration of the catalyst is in a range of 0.001 w/v % to 0.5 w/v %.

In some examples, a concentration of the catalyst is in a range of 0.001 w/v % to 0.2 w/v %.

In some examples of the reversible adhesive:
the reversible monomer of the reversible adhesive polymer includes NIPAM;
the acrylate cross-linker includes N,N-methylene bisacrylamide (BIS), a poly(ethylene glycol) diacrylate (PEG-DA), trimethylolpropane triacrylate (TMP-TA), a trimethylolpropane ethoxylate triacrylate, (TMPE-TA), poly(propylene glycol) diacrylate (PPG-DA), trimethylolpropane triacrylate, or a mixture thereof; and
the solvent includes dimethylformamide (DMF), water, ethanol, ethyl acetate, propylene carbonate, dimethyl sulfoxide (DMSO), propylene glycol, or a mixture thereof.

In some examples, the acrylate cross-linker includes a covalent diacrylate cross-linker, and wherein the covalent diacrylate cross-linker comprises N,N-methylene bisacrylamide (BIS), poly(ethylene glycol) diacrylate (PEG-DA), poly(propylene glycol) diacrylate (PPG-DA), or a mixture thereof.

In some examples, the acrylate cross-linker includes PEG-DA with an average molecular weight, Mn of about 250 kDa; PEG-DA with an average molecular weight, Mn of about 575 kDa; PEG-DA with an average molecular weight, Mn of about 700 kDa; PPG-DA with an average molecular weight Mn of about 800 kDa; TMPE-TA with an average molecular weight, Mn of about 428 kDa; TMPE-TA with an average molecular weight, Mn of about 912 kDa; or a mixture thereof.

In some examples, the polymer formulation further includes laponite, sodium polyacrylate, sodium alginate, tannic acid, chitosan, a silver particle, an aloe extract, propanediol, 1,4-butanediol, 1,5-pentanediol, hexanediol, octanediol, or a mixture thereof.

In some examples, the polymer formulation further includes an acrylate co-monomer; wherein the acrylate co-monomer comprises 2-ethylhexyl acrylate, di(ethylene glycol) 2-ethylhexyl acrylate, poly(ethylene glycol) methyletheracrylate (PEG-MEA), poly(propylene glycol) acrylate (PPG-A), sodium acrylate, 2-hydroxyethyl acrylate, 2-hydroxymethylethyl acrylate, dopamine acrylate, or a mixture thereof.

In some examples, the polymer formulation further includes vinyl acetate, 1-vinyl-2-pyrrolidinone, N-vinylcaprolactam, ethyl vinyl ether, isobutyl vinyl ether, propyl vinyl ether, butyl vinyl ether, cyclohexyl vinyl ether, tert-butyl vinyl ether, dodecylvinyl ether, 2-ethylhexyl vinyl ether, isopropyl vinyl ether, isobutyl vinyl ether, octyl vinyl ether, ethylene glycol vinyl ether, diethyl vinyl orthoformate, di(ethylene glycol) vinyl ether, 1,4-butanediol vinyl ether, 2-chloroethyl vinyl ether, or a mixture thereof.

In some examples, an adhesive strength of the reversible adhesive hydrogel mesh at a first temperature is higher than that of the reversible adhesive hydrogel mesh at a second temperature; and wherein the first temperature is higher than the second temperature.

In some examples, the adhesive strength of the reversible adhesive hydrogel mesh at the first temperature is in a range of 0.70 N/cm2 to 2.20 N/cm2, or 0.70 N/cm2 to 0.90 N/cm2; when the adhesive strength of the reversible adhesive hydrogel mesh is characterized using ASTM method F2258-05.

In some examples, the adhesive strength of the reversible adhesive hydrogel mesh at the second temperature is in a range of 0.40 N/cm2 to 0.90 N/cm2, or 0.50 N/cm2 to 0.80 N/cm2, or 0.53 N/cm2 to 0.70 N/cm2; when the adhesive strength of the reversible adhesive hydrogel mesh is characterized using ASTM method F2258-05.

In some examples, the adhesive strength of the reversible adhesive hydrogel mesh at the first temperature is in a range of 0.70 N/cm2 to 2.20 N/cm2, or 0.70 N/cm2 to 0.90 N/cm2; and wherein the adhesive strength of the reversible adhesive hydrogel mesh at the second temperature is in a range of 0.40 N/cm2 to 0.90 N/cm2, or 0.50 N/cm2 to 0.80 N/cm2, or 0.53 N/cm2 to 0.70 N/cm2; when the adhesive strength of the reversible adhesive hydrogel mesh is characterized using ASTM method F2258-05.

In some examples:
a concentration of the reversible monomer in the polymer formulation is in a range of 1 w/v % to 20 w/v %;
a concentration of acrylic acid (AA) in the polymer formulation is in a range of 1 w/v % to 20 w/v %; and
a concentration of the acrylate cross-linker in the polymer formulation is in a range of 0.1 w/v % to 5 w/v %.

In some examples:
a concentration of the reversible monomer in the polymer formulation is in a range of 5 w/v % to 15 w/v %;
a concentration of acrylic acid (AA) in the polymer formulation is in a range of 5 w/v % to 15 w/v %; and
a concentration of the acrylate cross-linker in the polymer formulation is in a range of 1 w/v % to 2 w/v %.

In some examples, the reversible monomer of the reversible adhesive polymer includes N-methyl-N-n-propylacrylamide, N-n-propylacrylamide, N-methyl-N-isopropylacrylamide, N-n-propylmethacrylamide, N-isopropylacrylamide (NIPAM), N-n-diethylacrylamide, N-isopropylmethacrylamide, N-cyclopropylacrylamide, N-ethylmethyacrylamide, N-methyl-N-ethylacrylamide, N-cyclopropylmethacrylamide, N-ethylacrylamide, N-n-diethylacrylamide, or a mixture thereof.

In some examples, the acrylate cross-linker includes a covalent diacrylate cross-linker, and wherein the covalent diacrylate cross-linker comprises N,N-methylene bisacrylamide (BIS), poly(ethylene glycol) diacrylate (PEG-DA), poly(propylene glycol) diacrylate (PPG-DA), or a mixture thereof.

In some examples, the acrylate cross-linker includes a bifunctional acrylate cross-linker, a trifunctional acrylate cross-linker, a tetrafunctional acrylate cross-linker, a hexafunctional acrylate cross-linker, or a mixture thereof.

In some examples, the acrylate cross-linker includes a bifunctional acrylate cross-linker, a trifunctional acrylate cross-linker, or a mixture thereof.

In some examples, the acrylate cross-linker includes a covalent diacrylate cross-linker, and wherein the covalent diacrylate cross-linker includes N,N-methylene bisacrylamide (BIS), poly(ethylene glycol) diacrylate (PEG-DA), poly(propylene glycol) diacrylate (PPG-DA), trimethylolpropane triacrylate, or a mixture thereof.

In some examples, the acrylate cross-linker includes PEG-DA with an average molecular weight, Mn of about 250 kDa; PEG-DA with an average molecular weight, Mn of about 575 kDa; PEG-DA with an average molecular weight, Mn of about 700 kDa; PPG-DA with an average molecular weight Mn of about 800 kDa; TMPE-TA with an average molecular weight, Mn of about 428 kDa; TMPE-TA with an average molecular weight, Mn of about 912 kDa; or a mixture thereof.

In some examples, the catalyst includes a photo-initiator for free radical polymerization.

In some examples, the catalyst includes potassium persulfate, azobisisobutyronitrile, 4,4′-azobis(4-cyanovaleric acid), 1,1′-azobis(cyclohexanecarbonitrile), 2-hydroxy-4′-(2-hydroxyethoxy)-2-methylpropiophenone, or a mixture thereof.

In some examples, the solvent includes dimethylformamide (DMF), water, ethanol, ethyl acetate, propylene carbonate, dimethyl sulfoxide (DMSO), propylene glycol, or a mixture thereof.

In some examples, the polymer formulation further includes an acrylate co-monomer; wherein the acrylate co-monomer comprises 2-ethylhexyl acrylate, di(ethylene glycol) 2-ethylhexyl acrylate, poly(ethylene glycol) methyletheracrylate (PEG-MEA), poly(propylene glycol) acrylate (PPG-A), sodium acrylate, 2-hydroxyethyl acrylate, 2-hydroxymethylethyl acrylate, dopamine acrylate, or a mixture thereof.

In some examples, the polymer formulation further includes a vinyl functionalized co-monomer.

In some examples, the polymer formulation further includes vinyl acetate, 1-vinyl-2-pyrrolidinone, N-vinylcaprolactam, ethyl vinyl ether, isobutyl vinyl ether, propyl vinyl ether, butyl vinyl ether, cyclohexyl vinyl ether, tert-butyl vinyl ether, dodecylvinyl ether, 2-ethylhexyl vinyl ether, isopropyl vinyl ether, isobutyl vinyl ether, octyl vinyl ether, ethylene glycol vinyl ether, diethyl vinyl orthoformate, di(ethylene glycol) vinyl ether, 1,4-butanediol vinyl ether, 2-chloroethyl vinyl ether, or a mixture thereof.

In some examples, the polymer formulation further includes laponite, sodium polyacrylate, sodium alginate, tannic acid, chitosan, a silver particle, an aloe extract, propanediol, 1,4-butanediol, 1,5-pentanediol, hexanediol, octanediol, or a mixture thereof.

In some examples, a lower critical solution temperature (LCST) of the reversible adhesive hydrogel mesh is in a range of 15° C. to 35° C.

In some examples, a concentration of the reversible monomer in the polymer formulation is in a range of 1 w/v % to 20 w/v %.

In some examples, a concentration of the reversible monomer in the polymer formulation is in a range of 5 w/v % to 15 w/v %.

In some examples, a concentration of acrylic acid (AA) in the polymer formulation is in a range of 1 w/v % to 20 w/v %.

In some examples, a concentration of acrylic acid in the polymer formulation is in a range of 5 w/v % to 15 w/v %.

In some examples, a concentration of the acrylate cross-linker in the polymer formulation is in a range of 0.1 w/v % to 5 w/v %.

In some examples, a concentration of the acrylate cross-linker in the polymer formulation is in a range of 1 w/v % to 2 w/v %.

In some examples, a concentration of the catalyst is in a range of 0.001 w/v % to 0.5 w/v %.

In some examples, a concentration of the catalyst is in a range of 0.001 w/v % to 0.2 w/v %.

In some examples, the adhesive strength of the reversible adhesive hydrogel mesh at a first temperature is higher than that of the reversible adhesive hydrogel mesh at a second temperature; and wherein the first temperature is higher than the second temperature.

In some examples, the adhesive strength of the reversible adhesive hydrogel mesh at the first temperature is in a range of 0.70 N/cm2 to 2.20 N/cm2, or 0.70 N/cm2 to 0.90 N/cm2; when the adhesive strength of the reversible adhesive hydrogel mesh is characterized using ASTM method F2258-05.

In some examples, the adhesive strength of the reversible adhesive hydrogel mesh at the second temperature is in a range of 0.40 N/cm2 to 0.90 N/cm2, or 0.50 N/cm2 to 0.80 N/cm2, or 0.53 N/cm2 to 0.70 N/cm2; when the adhesive strength of the reversible adhesive hydrogel mesh is characterized using ASTM method F2258-05.

In some examples, the adhesive strength of the reversible adhesive hydrogel mesh at the first temperature is in a range of 0.70 N/cm2 to 2.20 N/cm2, or 0.70 N/cm2 to 0.90 N/cm2; and wherein the adhesive strength of the reversible adhesive hydrogel mesh at the second temperature is in a range of 0.40 N/cm2 to 0.90 N/cm2, or 0.50 N/cm2 to 0.80 N/cm2, or 0.53 N/cm2 to 0.70 N/cm2; when the adhesive strength of the reversible adhesive hydrogel mesh is characterized using ASTM method F2258-05.

In some examples, the reversible adhesive hydrogel mesh is a reversible adhesive hydrogel mesh that adheres to a (human) tissue.

In some examples, the tissue is an epithelium.

In some examples, the tissue is a skin.

In some examples, the tissue is a skin and wherein the skin is a fragile skin.

In some examples, the skin is a fragile skin; wherein the fragile skin is a fragile skin of a human belonging to a pediatric population, a geriatric population, or a human with an injury or disease.

In some examples, the human is afflicted with a chronic wound, a burn injury, or a combination of any of these afflictions.

Some embodiments relate to a wound dressing, including a reversible adhesive hydrogel mesh as described herein.

In some examples, the wound dressing further includes a backing material; and wherein the reversible adhesive hydrogel mesh is formed on a surface of the backing material.

In some examples, the wound dressing further includes an adhesive primer layer placed between the reversible adhesive hydrogel mesh and the backing material.

In some examples, the wound dressing further includes a release liner material, wherein the release liner material is formed on a surface of the reversible adhesive hydrogel mesh.

Some embodiments relate to a method of preparation of a reversible adhesive hydrogel mesh, including:
preparing a reaction solution comprising a polymer formulation as described herein,
reacting the polymer formulation to prepare the reversible adhesive hydrogel mesh, and
treating the prepared reversible adhesive hydrogel mesh with a treatment solution comprising a humectant, and thereby
obtaining a reversible adhesive hydrogel mesh with improved tack.

In some examples of the method, the polymer formulation comprises:
a reversible monomer of a reversible adhesive polymer,
acrylic acid (AA),
an acrylate cross-linker,
a catalyst, and
a solvent.

In some examples, the treating the prepared reversible adhesive hydrogel mesh includes: treating the reversible adhesive hydrogel mesh with a first treatment solution before treating the prepared reversible adhesive hydrogel mesh with a treatment solution comprising a humectant, wherein the first treatment solution comprises water and an alcohol; and then treating the reversible adhesive hydrogel mesh with a second treatment solution, wherein the second treatment solution comprises a humectant and water.

In some examples, the treating the prepared reversible adhesive hydrogel mesh includes: treating the reversible adhesive hydrogel mesh with a treatment solution, wherein the treatment solution comprises a humectant, an alcohol and water; and thereby obtaining a reversible adhesive hydrogel mesh with improved tack.

In some examples, the treating the prepared reversible adhesive hydrogel mesh includes: treating the reversible adhesive hydrogel mesh with a treatment solution, wherein the treatment solution comprises a humectant and water; and thereby obtaining a reversible adhesive hydrogel mesh with improved tack.

In some examples, the humectant includes glycerol, ethylene glycol, propylene glycol, hexylene glycol, an aloe extract, hyaluronic acid, 2,3-butanediol, butyl ethyl propanediol, or a mixture thereof.

In some examples, the humectant concentration of a treatment solution is equal to or higher than 25 volume percent, equal to or higher than 50 volume percent, or equal to or higher than 75 volume percent in the mixture comprising a humectant and water.

Some examples relate to any combination of the reversible adhesive hydrogel meshes, wound dressings or methods of preparation disclosed herein.

### BRIEF DESCRIPTION OF DRAWINGS

The drawings are of illustrative examples. They do not illustrate all examples. Other examples may be used in addition or instead. Details that may be apparent or unnecessary may be omitted to save space or for more effective illustration. Some examples may be practiced with additional components or steps and/or without all of the components or steps that are illustrated. When the same numeral appears in different drawings, it refers to the same or like components or steps.

FIG. 1. a) Diagram representing the construction of a reactor. Gaskets of varied thicknesses are used to set desired thickness of mesh; b) Photo of reactor in light box.

FIG. 2. Diagram representing the rheological measurement of adhesive mesh under various temperatures. The LCST may be identified using this methodology.

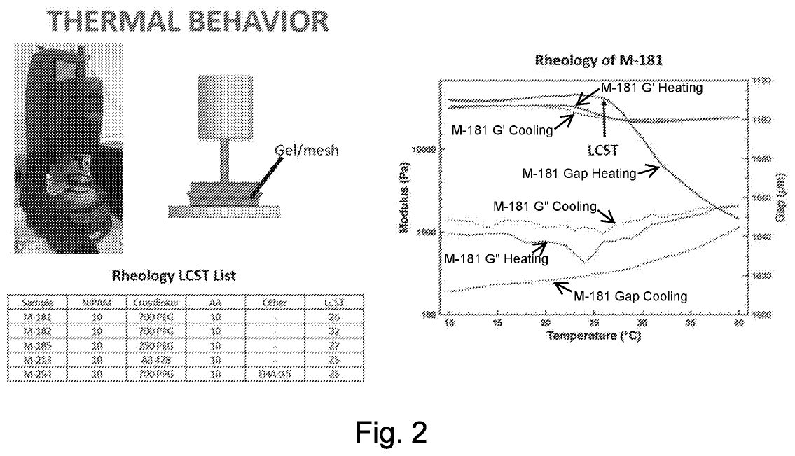

### DETAILED DESCRIPTION OF ILLUSTRATIVE EXAMPLES

Illustrative examples are now described. Other examples may be used in addition or instead. Details that may be apparent or unnecessary may be omitted to save space or for a more effective presentation. Some examples may be practiced with additional components or steps and/or without all of the components or steps that are described.

In this disclosure, the following acronyms and abbreviations are used.

250 PEG-DA: Poly(ethylene glycol) diacrylate, Mn=250 kDa

428 TMPE-TA: Trimethylolpropane ethoxylate triacrylate, Mn=428 kDa

575 PEG-DA: Poly(ethylene glycol) diacrylate, Mn=575 kDa

700 PEG-DA: Poly(ethylene glycol) diacrylate, Mn=700 kDa

800 PPG-DA: Poly(propylene glycol) diacrylate, Mn=800 kDa

912 TMPE-TA: Trimethylolpropane ethoxylate triacrylate, Mn=912 kDa

AA: acrylic acid

BIS: N,N′-methylene bisacrylamide

CASRN: Chemical Abstracts Service Registry Number

DI-H2O: Distilled Water

DMF: dimethylformamide

DMSO: dimethylsulfoxide

DPE-HA: Dipentaerythritol penta-/hexa-acrylate

EHA: 2-ethylhexyl acrylate

NIPAM: N-isopropylacrylamide

PE-TA: Pentaerythritol tetraacrylate

PPG-DA: Poly(propylene glycol) diacrylate

TMP-TA: Trimethylolpropane triacrylate

This disclosure relates generally to adhesives and particularly to reversible adhesives. This disclosure also relates to reversible adhesive hydrogel meshes. This disclosure also relates to polymer formulations that may be used in preparation of the reversible adhesive hydrogel meshes. The polymer formulations may comprise a reversible monomer of a reversible adhesive polymer, acrylic acid (AA), an acrylate cross-linker, a photo-initiator for free radical polymerization, and a solvent. This disclosure also relates to a wound dressing comprising the reversible adhesive hydrogel meshes. Such wound dressings are particularly suitable for treatment of damaged sensitive tissue, for example, wounds formed on a fragile skin.

In one example, the reversible adhesive hydrogel meshes of this disclosure may have improved adhesive properties or higher adhesiveness at a first predetermined condition than at a second predetermined condition.

For example, these reversible adhesive hydrogel meshes may provide sufficient adhesiveness at or above a skin temperature such that a wound dressing incorporating such reversible adhesives properly may adheres to skin, for example at a temperature in a range of 35° C.-41° C. (i.e. the first predetermined condition). This level of adhesiveness at or above a skin temperature may be required so that, for example, the wound dressing remains adhered to the skin surrounding the wound to allow the wound to heal within a reasonable time. When this wound dressing is cooled down for example, below the skin temperature, by using, for example, ice (i.e. the second predetermined condition), the adhesiveness of the dressing may thereby substantially be reduced to a level that the dressing may be removed from the skin with negligible force. This level of adhesiveness at a temperature below the skin temperature may be required so that, for example, the wound dressing may easily be removed from the skin surrounding the wound with minimal damage to the skin and/or the wound and/or minimal pain. That is, in one example, adhesiveness of this reversible adhesive hydrogel mesh at about 37° C. may substantially be higher than its adhesiveness below 37° C. In this example, the adhesive may be thermally reversible at about 37° C. However, depending on type of its application, the reversible adhesive hydrogel mesh may be thermally reversible at any other temperature.

This reversibility may be desired: it may be turned on or off at will making it suitable for wide variety of applications where reversibility of adhesiveness is desired or even required.

The thermal reversibility may not be the only mechanism by which the reversible adhesives may be manufactured. The reversibility of such adhesives may also be controlled by using other mechanisms. For example, such adhesives may provide sufficient adherence to a substrate at normal lighting conditions (e.g. sun or artificial lights). But, their adhesiveness may be reduced to a negligible level when they are irradiated by an ultraviolet (UV) light. In another example, they may provide sufficient adhesiveness to tissue at normal humidity conditions (e.g. skin humidity or weather humidity). However, they may lose their adhesiveness when sufficient amount of solvent (e.g. water, alcohol and the like) is applied. The water applied, for example, may contain ionic or non-ionic solutes. All such reversible adhesives are within the scope of this disclosure. Such reversible adhesives in this disclosure is referred to as “reversible adhesive hydrogel meshes” or “hydrogel meshes”.

The reversible adhesive hydrogel meshes of the instant disclosure may be suitable in binding any two surfaces together, for example a wood surface to a glass surface. The reversible adhesive hydrogel meshes may be particularly suitable in binding a wound dressing to a tissue. The tissue may be a human tissue or a tissue of a non-human organism such as another mammal, vertebrate or microorganism. The tissue may be a living or dead cell culture. The tissue may be in any condition, e.g. it can be wet or dry. In one example, the tissue is skin, which may be the soft outer covering of an animal, open wound or combinations thereof. That is, the reversible adhesive hydrogel meshes may provide adhesion to a skin, a wound formed on a skin or both the skin and the open wound. In another example, the skin may be a fragile skin. Age-related changes in skin morphology in the elderly may result in the development of fragile skin. With age the outer skin layer (epidermis) may become thinner, with decreases in extracellular components, such as collagen and elastin, which may lead to decrease in tensile strength and elasticity of the skin. Other age-related skin changes may include thinning of the subcutaneous fat layer, increased blood vessel fragility and a decrease in the adhesiveness between the dermis and the underlying loose connective tissues, resulting in increased vulnerability to skin tears and ruptures. Fragile skin may also be induced by cancer chemo- and radiation therapy. Humans with fragile skin may be prone to have wounds caused by strains inflicted on such skins at levels negligible to normal human skin. For example, a soft impact on a fragile skin by an object can easily induce a wound on a fragile skin. If such wound may be covered by a commercially available typical wound dressing (e.g. an adhesive bandage) for protective or treatment purposes, the removal of the wound dressing later may become an important problem due to considerable adherence of the wound dressing to the fragile skin. The wound dressing removal may easily cause further damage to the fragile skin or to the wound formed on such skin.

The reversible adhesive hydrogel meshes of the instant disclosure may provide solutions for this important problem. The wound dressings manufactured by using the reversible adhesive hydrogel meshes may adhere to fragile skin at skin temperatures and may easily be removed with minimal force and negligible or no further damage to the skin when the wound dressing is cooled below the skin temperature, for example by using cold air, cooled compresses, or ice.

Although, the wound dressings of the instant disclosure may be explained above by way of the fragile skin example, they may be used in treatment of any type of wound. And all such applications are within the scope of this disclosure.

The reversible adhesive hydrogel mesh's adhesiveness may be obtained in part by using conventional adhesives such as pressure sensitive adhesives or chemical compounds used in manufacturing such conventional adhesives, but their adhesiveness is controlled or turned on or off by incorporation of reversible adhesives or chemical compounds used in manufacturing of such reversible adhesives to chemical structure or formulation of the conventional adhesives, as explained below.

The reversible adhesive hydrogel mesh may have novel thermal behavior in aqueous media: they may have inverse solubility with increasing temperature. Their molecular structure may transition from a hydrophilic to a hydrophobic structure by heating, causing them to aggregate at a higher temperature while they may be completely soluble at a lower temperature. This structure change may happen rather abruptly at a temperature that is known as the lower critical solution temperature (LCST). For example, while a reversible adhesive hydrogel meshes comprising poly(N-isopropylacrylamide) may be hydrophilic at a temperature below LCST, it becomes hydrophobic above LCST and extrudes aqueous media which was previously absorbed inside the mesh. For this thermally reversible polymer, LCST is in the range of 30° C. to 35° C. This polymer is adhesive to the tissue above LCST and has substantially lowered or even negligible adhesiveness below LCST.

There are many reversible polymers that can be used to prepare the reversible adhesive hydrogel meshes. Their LCST may change together with their molecular structure. Copolymers of a thermally reversible polymer with other thermally reversible polymer or any other polymer may also be prepared to obtain polymers with varying LCSTs. Thereby, LCST may be controlled at a desired level by having variety of homopolymers and copolymers and numerous reversible adhesives may be obtained for wide variety of medical or non-medical applications. All such homopolymers and copolymers are within the scope of this disclosure.

In one example, the reversible adhesive hydrogel meshes may be thermally reversible at a temperature within the range of 0° C. to 100° C. In another example, the reversible adhesive hydrogel mesh may thermally be reversible at a temperature within the range of 0° C. to 50° C.

Examples of (thermally) reversible adhesive polymers and their typical LCSTs are poly(N-methyl-N-n-propylacrylamide), about 19.8° C.; poly(N-n-propylacrylamide), about 21.5° C.; poly(N-methyl-N-isopropylacrylamide) about 22.3° C.; poly(N-n-propylmethacrylamide), about 28.0° C.; poly(N-isopropylacrylamide), about 30.9°; poly(N, n-diethylacrylamide), about 32.0° C.; poly(N-isopropylmethacrylamide), about 44.0° C.; poly(N-cyclopropylacrylamide), about 45.5° C.; poly(N-ethylmethyacrylamide), about 50.0° C.; poly(N-methyl-N-ethylacrylamide), about 56.0° C.; poly(N-cyclopropylmethacrylamide), about 59.0° C.; and poly(N-ethylacrylamide), about 72.0° C., and their co-polymers with other polymers, and mixtures thereof. Another example of a thermally reversible polymer is acrylate-modified tri-block copolymer of polyethylene oxide (PEO)-co-poly(p-phenylene oxide) (PPO)-co-polyethylene glycol (PEO). In the last example, the molecular ratio of each polymer can be varied to vary the LCST of the polymer. Examples of reversible monomers that may be used for the purposes of the instant disclosure are the monomers used in preparation of such thermally reversible polymers.

In one example, the thermally reversible polymers may be polymers prepared by polymerization of monomers of N-alkylacrylamide, N-alkylmethacrylamide or mixtures thereof. One example of such monomers may be N-isopropylacrylamide. And one example of such polymer may be poly(N-isopropylacrylamide).

The wound dressings may comprise the reversible adhesive hydrogel meshes to provide reversible wound dressings. In one example, the wound dressings may comprise the thermally reversible adhesive to provide the thermally reversible wound dressing.

The wound dressings may further comprise a substrate (or backing material). These substrates may have variety of shapes and structures to carry the reversible adhesive. For example the substrate may be substantially flat with relatively smooth surfaces, like polymer films; it may have a sponge like structure; and it may also have surfaces comprising filamentary structures.

Examples of substrates are cloths, meshes or films. These substrates may have variety of shapes. Examples of cloths include woven cloths such as gauze, non-woven cloths, fabrics, sponges, or composites thereof. Examples of films include films manufactured by using polymers such as polyethylene, polyester, polyurethane, silicone, polyimide, poly(monochloro-p-xylylene) (e.g. parylene C), poly(dimethylsiloxane) (e.g. PDMS) or films manufactured by using biologically derived materials such as elastin, alginates, chitin, collagen and fibrin. Polypeptides derived from biologic materials such as elastin may also be used. Composites of all such materials may also be used to manufacture the substrates and are thereby within the scope of this disclosure.

Gauze, non-woven cloths, fabrics and/or the like may be manufactured by using fibers such as natural fibers, synthetic fibers and composites thereof. These fibers may comprise, for example, cotton, linen, jute, hemp, cotton, wool, wood pulp, regenerated cellulosic fibers such as viscose rayon and cuprammonium rayon, modified cellulosic fibers such as cellulose acetate, synthetic fibers such as those derived from polyesters, polyamides, polyacrylics, biocompatible/biodegradable fibers such as polylactone, or composites thereof.

These substrates may be substrates used for variety of applications. For example, they may be used as surgical barriers, surgical patches (e.g., dural patches), surgical wraps (e.g., vascular, perivascular, adventitial, periadventitital wraps, and adventitial sheets), surgical dressings, meshes (e.g., perivascular meshes), bandages, tapes, tissue coverings and the like.

Examples of such substrates further include polyester, polyurethane, silicone sheet, polypropylene (PP), polyethylene (PE), polyvinyl chloride (PVC) and composites thereof. One example of the polyester polyethylene terephthalate (PET). Commercial examples of polyester films are Mylar or perforated Telfa films.

The substrate surface may be plasma treated or chemically treated to improve its bonding with the reversible adhesive. For example, such treatments may allow the attachment of vinyl bonds or functional groups to the substrate surface. In one example, the reversible adhesive hydrogel meshes may be cohesively or chemically bonded to the substrate. In another example, the reversible adhesive hydrogel meshes may further comprise a chemical compound to improve the adhesion of the reversible adhesive to the substrate. This chemical compound, for example, may be a so-called adhesion promoter. Yet, in another example, the wound dressing may further comprise an intermediary adhesive layer between the substrate and the reversible adhesive to improve adhesion of the reversible adhesive to the substrate. For example, this intermediary adhesive layer or primer may comprise a monomer or polymer of this monomer. For example, this monomer may be a so-called adhesion promoter.

In one example, at least one surface of the substrate is partially or completely covered with the reversible adhesive hydrogel mesh. The remaining surface that is not covered with the reversible adhesive hydrogel mesh may be covered with another material, for example with gauze.

Although the reversible adhesive hydrogel meshes are described above by way of medical applications, these adhesives may be suitable for applications in other fields. For example, electronic, optical, electro-optical components or even automotive components, which need repairs, replacements or repositioning, may benefit from the reversible adhesives or reversible adhesive tapes manufactured by using such adhesives.

In one example, the reversible wound dressings are thermally reversible at a temperature within the range of 0° C. to 100° C. In another example, the reversible wound dressings are thermally reversible at a temperature within the range of 0° C. to 50° C.

In one example, the reversible adhesive hydrogel mesh may be prepared by reacting a polymer formulation. The polymer formulation may include a reversible monomer of a reversible adhesive polymer, acrylic acid (AA), an acrylate cross-linker, a photo-initiator for free radical polymerization, and a solvent. In one example, the polymer formulation may include N-isopropylacrylamide (NIPAM), acrylic acid (AA), a covalent diacrylate cross-linker or a covalent triacrylate cross-linker, a photo-initiator for free radical polymerization, and dimethylsulfoxide. In one example, the polymer formulation may include N-isopropylacrylamide (NIPAM), acrylic acid (AA), a covalent diacrylate cross-linker or a covalent triacrylate cross-linker, a photo-initiator for free radical polymerization, and propylene glycol.

The reversible monomer of a reversible adhesive polymer may comprise N-methyl-N-n-propylacrylamide, N-n-propylacrylamide, N-methyl-N-isopropylacrylamide, N-n-propylmethacrylamide, N-isopropylacrylamide, N-n-diethylacrylamide, N-isopropylmethacrylamide, N-cyclopropylacrylamide, N-ethylmethyacrylamide, N-methyl-N-ethylacrylamide, N-cyclopropylmethacrylamide, N-ethylacrylamide, N-n-diethylacrylamide, or a mixture thereof.

The acrylate cross-linker may include a multifunctional acrylate cross-linker. Examples of the multifunctional acrylate cross-linker may be a bifunctional acrylate cross-linker, a trifunctional acrylate cross-linker, a tetrafunctional acrylate cross-linker, a hexafunctional acrylate cross-linker, or a mixture thereof. Examples of the acrylate cross-linker may be N,N-methylene bisacrylamide (BIS), a poly(ethylene glycol) diacrylate (PEG-DA), trimethylolpropane triacrylate (TMP-TA), a trimethylolpropane ethoxylate triacrylate, (TMPE-TA), or a mixture thereof. Examples of the acrylate cross-linker may be a covalent diacrylate cross-linker, and wherein the covalent diacrylate cross-linker may be N,N-methylene bisacrylamide (BIS), poly(ethylene glycol) diacrylate (PEG-DA), poly(propylene glycol) diacrylate (PPG-DA) or a mixture thereof. Examples of the poly(ethylene glycol) diacrylate (PEG-DA) may be PEG-DA with an average molecular weight, Mn of 250 kDa; PEG-DA with an average molecular weight, Mn of 575 kDa; PEG-DA with an average molecular weight, Mn of 700 kDa, or a mixture thereof. An example of poly(propylene glycol) diacrylate (PPG-DA) may be PPG-DA with an average molecular weight Mn of 800 kDa. Examples of the trimethylolpropane ethoxylate triacrylate, (TMPE-TA) may be TMPE-TA with an average molecular weight, Mn of 428 kDa; TMPE-TA with an average molecular weight, Mn of 912 kDa, or a mixture thereof. An example of the acrylate cross-linker may be trimethylolpropane triacrylate.

In one example, the polymer formulation may further include an acrylate comonomer. Examples of the acrylate comonomer may include 2-ethylhexyl acrylate, di(ethylene glycol) 2-ethylhexyl acrylate, poly(ethylene glycol) methyletheracrylate (PEG-MEA), poly(propylene glycol) acrylate, sodium acrylate, 2-hydroxyethyl acrylate, 2-hydroxymethylethyl acrylate, dopamine acrylate, or a mixture thereof.

In one example, the polymer formulation may further include an additive. Examples of the additive may be laponite, sodium polyacrylate, sodium alginate, tannic acid, chitosan, a silver particle, an aloe extract, propanediol, 1,4-butanediol, 1,5-pentanediol, hexanediol, octanediol, or a mixture thereof.

In one example, the solvent may include dimethylformamide (DMF), water, ethanol, ethyl acetate, propylene carbonate, dimethyl sulfoxide (DMSO), propylene glycol, or a mixture thereof. In another example, the solvent my include dimethyl sulfoxide (DMSO).

In one example, a lower critical solution temperature (LCST) of the reversible adhesive hydrogel mesh may be in a range of 15° C. to 35° C.

In one example, a concentration of the reversible monomer of a reversible adhesive polymer in the polymer formulation may be in a range of 1 w/v % to 20 w/v %. In another example, a concentration of the reversible monomer of a reversible adhesive polymer in the polymer formulation may be in a range of 5 w/v % to 15 w/v %.

In one example, a concentration of acrylic acid in the polymer formulation may be in a range of 1 w/v % to 20 w/v %. In another example, a concentration of acrylic acid in the polymer formulation may be in a range of 5 w/v % to 15 w/v %.

In one example, a concentration of the acrylate cross linker in the polymer formulation may be in a range of 0.1 w/v % to 5 w/v %. In another example, a concentration of the acrylate cross linker in the polymer formulation may be in a range of 1 w/v % to 2 w/v %.

In one example, the photo-initiator may be 2-hydroxy-4′-(2-hydroxyethoxy)-2-methylpropiophenone.

In one example, the reversible adhesive hydrogel mesh may be a reversible adhesive hydrogel mesh that adheres to a (human) tissue. The tissue may comprise any tissue. For example, the tissue may comprise epithelial tissue (epithelium), connective tissue, muscle tissue, nervous tissue, or a composite thereof. For example, the tissue may comprise an epithelial tissue. The epithelial tissue may, for example, be a tissue that lines outer surfaces of organs, blood vessels, and inner surfaces of cavities of internal organs throughout a (human) body. An example of the tissue may be a skin. The skin may comprise epidermis, basement membrane, dermis, subcutaneous tissue, or a composite thereof. The skin may be a fragile skin. The fragile skin may be a fragile skin of a human belonging to a pediatric population, a geriatric population, or a human with an injury or disease. The human with an injury or disease may be afflicted with a chronic wound (e.g. a ulcer), a burn injury, or a combination of any of these afflictions.

This disclosure also relates to a wound dressing. This wound dressing may include any reversible adhesive hydrogel mesh of this disclosure. The wound dressing may further include a backing material.

This disclosure also relates to a method of preparation of a reversible adhesive hydrogel mesh. This method may include preparing a reaction solution comprising any polymer formulation of this disclosure, reacting the polymer formulation to prepare the reversible adhesive hydrogel mesh, and treating the prepared reversible adhesive hydrogel mesh with a humectant. Examples of the humectant may include glycerol, ethylene glycol, propylene glycol, hexylene glycol, an aloe extract, hyaluronic acid, 2,3-butanediol, butyl ethyl propanediol, or a mixture thereof. Method of the treating the prepared reversible adhesive hydrogel mesh with a humectant may include washing the reversible adhesive hydrogel mesh with a hydrogel wash solvent (“a first washing”), washing the reversible adhesive hydrogel mesh with a mixture comprising a hydrogel wash solvent and a humectant (“a second washing”) after the first washing, washing the reversible adhesive hydrogel mesh with a mixture comprising a hydrogel wash solvent, a humectant, and water (“a third washing”) after the second washing, and washing the reversible adhesive hydrogel mesh with a mixture comprising a humectant and water (“a fourth washing”) after the third washing, and thereby obtaining a reversible adhesive hydrogel mesh with improved tack. In the fourth wash, the humectant concentration may be equal to or higher than 25 volume percent, equal to or higher than 50 volume percent, or equal to or higher than 75 volume percent in the mixture comprising a humectant and water. In one example, the method of treating the prepared reversible adhesive hydrogel mesh with a humectant may include washing the reversible adhesive hydrogel mesh a mixture comprising humectant and water (“a first washing”) and washing the reversible adhesive hydrogel mesh with a mixture comprising humectant and water (“a second washing”). In the first and second wash, the humectant concentration may be equal to or higher than 25 volume percent, equal to or higher than 50 volume percent, or equal to or higher than 75 volume percent in the mixture comprising a humectant and water.

### Example 1. General Reaction Conditions and Procedure

In this disclosure, adhesive hydrogel meshes may be synthesized by photo initiated free radical polymerization. Each adhesive hydrogel mesh may be synthesized using different sizes of the reaction vessels ranging in area from 6×6″ squares to 10″×10″ squares with thicknesses ranging from one 1/16″ to 1/64″.

In this disclosure, all reagents are used as received from suppliers without further purification or modifications, CASRN and P/N listed in Table 1. Acrylic acid, acrylate cross-linkers, acrylate comonomers, and 2-hydroxy-4′-(2-hydroxyethoxy)-2-methylpropiophenone are purchased from Sigma-Aldrich. N-isopropylacrylamide was purchased from Acros. Dimethylsulfoxide was purchased from VWR Chemicals. Ethanol was purchased from Koptec. Distilled water (DI-H2O), filtered by MilliQ, was prepared in house.

TABLE 1
Chemicals Used in Preparation of Hydrogel Meshes

| | | | P/N | |
|---|---|---|---|---|
| Reagent | Class | CASRN | or SKU | Supplier |
| Acrylic acid | Monomer | 79-10-7 | 147230 | Sigma- |
| | | | | Aldrich |
| 2-hydroxy-4′-(2- | Photo- | 106797-53-9 | 410896 | Sigma- |
| hydroxyethoxy)-2- | initiator | | | Aldrich |
| methylpropiophenone | | | | |
| N-isopropyl- | Monomer | 2210-25-5 | A0368933 | Acros |
| acrylamide | | | | |
| Dimethylsulfoxide | Solvent | 67-68-5 | BDH1115 | VWR |
| | | | | Chemicals |
| Dimethylformamide^{†} | Solvent | 68-12-2 | BDH117 | VWR |
| | | | | Chemicals |
| Ethanol, 200 proof | Solvent | 64-17-5 | V1001 | Koptec |
| Propylene Carbonate^{†} | Solvent | 108-32-7 | A15552 | Alfa |
| | | | | Aesar |
| Ethylene Carbonate^{†} | Solvent | 96-49-1 | 676802 | Sigma- |
| | | | | Aldrich |
| Propylene Glycol | Solvent | 57-55-6 | W294004 | Sigma- |
| | | | | Aldrich |
| 2-ethylhexyl acrylate | Co- | 103-11-7 | 290815 | Sigma- |
| | monomer | | | Aldirch |
| Di(ethylene glycol) 2- | Co- | 117646-83-0 | 407542 | Sigma- |
| ethylhexyl acrylate | monomer | | | Aldrich |
| Poly(ethylene glycol) | Co- | 32171-39-4 | 454990 | Sigma- |
| methyletheracrylate | monomer | | | Aldrich |
| Poly(propylene glycol) | Co- | 50858-51-0 | 469815 | Sigma- |
| acrylate | monomer | | | Aldrich |
| Sodium acrylate | Co- | 7446-81-3 | 408220 | Sigma- |
| | monomer | | | Aldrich |
| 2-hydroxyethyl | Co- | 818-61-1 | 292818 | Sigma- |
| acrylate | monomer | | | Aldrich |
| 2-hydroxymethylethyl | Co- | 868-77-9 | 477028 | Sigma- |
| acrylate | monomer | | | Aldrich |
| N,N-methylene | Cross- | 110-26-9 | M-7279 | Sigma- |
| bisacrylamide | linker, | | | Aldrich |
| | diacrylate | | | |
| Poly(ethylene glycol) | Cross- | 26570-48-9 | 475629 | Sigma- |
| diacrylate, Mn = 250 | linker, | | | Aldrich |
| kDa | diacrylate | | | |
| Poly(ethylene glycol) | Cross- | 26570-48-9 | 437441 | Sigma- |
| diacrylate, Mn = 575 | linker, | | | Aldrich |
| kDa | diacrylate | | | |
| Poly(ethylene glycol) | Cross- | 26570-48-9 | 455008 | Sigma- |
| diacrylate, Mn = 700 | linker, | | | Aldrich |
| kDa | diacrylate | | | |
| Poly(propylene glycol) | Cross- | 52496-08-9 | 455024 | Sigma- |
| diacrylate, Mn = 800 | linker, | | | Aldrich |
| | diacrylate | | | |
| Trimethylolpropane | Cross- | 15625-89-5 | 246808 | Sigma- |
| triacrylate | linker, | | | Aldrich |
| | triacrylate | | | |
| Trimethylolpropane | Cross- | 28961-43-5 | 409073 | Sigma- |
| ethoxylate triacrylate, | linker, | | | Aldrich |
| Mn = 428 kDa | triacrylate | | | |
| Trimethylolpropane | Cross- | 28961-43-5 | 412198 | Sigma- |
| ethoxylate triacrylate, | linker, | | | Aldirch |
| Mn = 912 kDa | triacrylate | | | |
| Pentaerythritol | Cross- | 4986-89-4 | 408263 | Sigma- |
| tetraacrylate | linker, | | | Aldrich |
| | tetra- | | | |
| | acrylate | | | |
| Dipentaerythritol | Cross- | 60506-81-2 | 407283 | Sigma- |
| penta-/hexa-acrylate | linker, | | | Aldrich |
| | hexa- | | | |
| | acrylate | | | |
| Laponite | Additive | | | Southern |
| | | | | Clay |
| | | | | Products |
| Sodium Polyacrylate | Additive | 9003-04-7 | 432784 | Sigma- |
| | | | | Aldrich |
| Sodium Alginate | Additive | 9005-38-3 | W201502 | Sigma- |
| | | | | Aldrich |
| Chitosan | Additive | 9012-76-4 | 448877 | Sigma- |
| | | | | Aldrich |

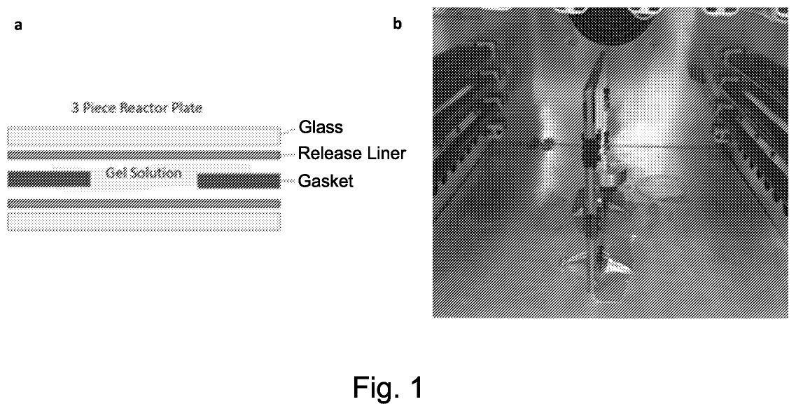

Acrylic acid, NIPAM, cross-linker, additives (if used), and co-monomer (if used) are dissolved in a solvent in an amber glass bottle. An example of the solvent is dimethylsulfoxide. An another example of the solvent is propylene glycol. The photo-initiator is added to the reaction solution last, followed by a nitrogen gas sparge. The finished reaction solution is transferred to a reactor, shown in FIG. 1, of desired size (about 6″ to about 10″ square) and thickness (about 1/16 to about 1/64″) via syringe. The reactor assembly is irradiated with light with a wavelength maximum of about 360 nm in a nitrogen flushed light box for about 30 minutes. The synthetic mesh is then transferred to a shallow dish, where it is submerged in ethanol (200 proof) and gently rocked for about 15 minutes. The mesh is subsequently submerged and rocked in about 150 proof, about 100 proof, and about 50 proof aqueous ethanol solutions for about 15 minutes each, pouring off the solutions between each wash. Finally, the mesh is washed in distilled water two consecutive times. The finished mesh is stored in a distilled water in a sealed container. Alternatively, the synthetic mesh can be washed by a different series of steps: the synthetic mesh is then transferred to a shallow dish, where it is submerged in a humectant and water mixture and gently rocked for about 15 minutes. The mesh is subsequently submerged in a fresh humectant and water mixture and gently rocked for about 15 minutes. The washed mesh is dried in air. The finished mesh is stored between two pieces of release liner.

### Example 2. Solvent Choice

Seven solvents have been screened for hydrogel mesh synthesis: dimethylformamide (DMF), water, ethanol, ethyl acetate, propylene carbonate, propylene glycol, and dimethyl sulfoxide (DMSO). Polymerization of some polymer formulations, which included water, ethanol, or a mixture thereof as solvents, has resulted in incomplete polymerization and poorly formed fragile meshes. Similarly, polymerization of some polymer formulations, which included polar solvents, ethyl acetate and propylene carbonate, has resulted in incomplete polymerization and poorly formed fragile meshes. Because DMSO is known to be a chemical used for topical application for conditions like arthritis and muscle aches, the hydrogel meshes made by using this solvent may potentially be more biocompatible.

### Example 3. Exemplary Polymer Formulations

Typical polymer formulations included N-isopropylacrylamide (NIPAM), acrylic acid (AA), and N,N-methylene bisacrylamide (BIS), poly(ethylene glycol) diacrylate (PEG-DA, 700 Mn), poly(propylene glycol) diacrylate (PPG-DA, 800 Mn) as a covalent diacrylate cross-linker. NIPAM may primarily be responsible for imparting thermal responsiveness to the mesh, whereas AA may impart adhesive strength. Together, NIPAM and AA repeat units in polymer chains may have hydrogen bonding intramolecular forces, which may influence mesh elasticity and cohesive strength. Identity of the covalent cross-linker may also play a substantial role on physical properties of meshes, which is discussed in a later section.

Weight per volume ratios (w/v %) were varied in a range of 5% to 15% for both NIPAM and AA, and in a range of 0.25% to 2% for cross-linkers (Table 2). In some formulations polymer formulations included about 10% NIPAM, about 10% AA, and about 1% cross-linker. Resulting meshes have good adhesion, elasticity, and observable temperature transitions. Upon dropping either NIPAM or AA concentration to about 5% w/v; adhesion, elasticity, and/or temperature transitions of the hydrogel meshes decreased or lost entirely. Likewise, increasing NIPAM or AA concentration to about 15% w/v results in similar loss of favorable properties. Similarly, low (<1%) or high (>1%) loadings of diacrylate cross-linker led to fragile, or brittle, non-elastic meshes.

TABLE 2
Exemplary polymer formulations.

| Formu- | NIPAM | AA | Cross-linker | Cross-linker |
|---|---|---|---|---|
| lation | (% w/v) | (% w/v) | Identity | (% w/v) |
| M-096 | 10 | 10 | BIS | 0.25 |
| M-102 | 10 | 10 | BIS | 0.5 |
| M-105 | 10 | 10 | BIS | 1 |
| M-123 | 5 | 10 | BIS | 1 |
| M-124 | 1 | 10 | BIS | 1 |
| M-144 | 10 | 10 | PEG-DA, 700 Mn | 1.1 |
| M-145 | 10 | 10 | PEG-DA, 700 Mn | 1.2 |
| M-172 | 5 | 10 | PEG-DA, 700 Mn | 1 |
| M-174 | 10 | 0 | PEG-DA, 700 Mn | 1 |
| M-176 | 10 | 0 | BIS | 1 |
| M-179 | 10 | 15 | PEG-DA, 700 Mn | 1 |
| M-181 | 10 | 10 | PEG-DA, 700 Mn | 1 |
| M-182 | 10 | 10 | PPG-DA, 800 Mn | 1 |
| M-195 | 10 | 5 | PEG-DA, 700 Mn | 1 |
| M-196 | 10 | 6 | PEG-DA, 700 Mn | 1 |
| M-197 | 10 | 7 | PEG-DA, 700 Mn | 1 |
| M-198 | 10 | 8 | PEG-DA, 700 Mn | 1 |
| M-199 | 10 | 9 | PEG-DA, 700 Mn | 1 |
| M-200 | 10 | 11 | PEG-DA, 700 Mn | 1 |
| M-201 | 10 | 12 | PEG-DA, 700 Mn | 1 |
| M-202 | 10 | 13 | PEG-DA, 700 Mn | 1 |
| M-203 | 10 | 14 | PEG-DA, 700 Mn | 1 |
| M-204 | 6 | 10 | PEG-DA, 700 Mn | 1 |
| M-205 | 7 | 10 | PEG-DA, 700 Mn | 1 |
| M-206 | 8 | 10 | PEG-DA, 700 Mn | 1 |
| M-207 | 9 | 10 | PEG-DA, 700 Mn | 1 |
| M-208 | 11 | 10 | PEG-DA, 700 Mn | 1 |
| M-209 | 12 | 10 | PEG-DA, 700 Mn | 1 |
| M-210 | 13 | 10 | PEG-DA, 700 Mn | 1 |
| M-211 | 14 | 10 | PEG-DA, 700 Mn | 1 |
| M-212 | 15 | 10 | PEG-DA, 700 Mn | 1 |
| M-214 | 10 | 10 | TMP-TA | 1 |
| M-261 | 10 | 10 | PEG-DA, 700 Mn | 2.8 |
| M-280 | 5 | 10 | PPG-DA, 800 Mn | 1 |
| M-281 | 15 | 10 | PPG-DA, 800 Mn | 1 |
| M-282 | 10 | 5 | PPG-DA, 800 Mn | 1 |
| M-283 | 10 | 15 | PPG-DA, 800 Mn | 1 |
| M-286 | 8 | 10 | TMP-TA | 1 |
| M-287 | 12 | 10 | TMP-TA | 1 |
| M-288 | 15 | 10 | TMP-TA | 1 |
| M-293 | 9 | 10 | TMP-TA | 1 |
| M-294 | 11 | 10 | TMP-TA | 1 |
| M-295 | 13 | 10 | TMP-TA | 1 |
| M-296 | 14 | 10 | TMP-TA | 1 |
| M-297 | 10 | 9 | TMP-TA | 1 |
| M-298 | 10 | 11 | TMP-TA | 1 |
| M-299 | 10 | 8 | TMP-TA | 1 |
| M-300 | 10 | 12 | TMP-TA | 1 |
| M-301 | 10 | 7 | TMP-TA | 1 |
| M-302 | 10 | 13 | TMP-TA | 1 |

### Example 4. Covalent Cross-Linkers

Covalent cross-linkers may act as physical bridges between long polymer chains in hydrogel meshes. Without a cross-linking agent, the polymers may dissolve in solvents rather than retaining a semi-solid gel form. Cross-linkers may influence mesh elasticity, brittleness, overall surface energy, and the degree to which a mesh swells in solvents. Cross-linkers may be bifunctional (e.g. diacrylates), containing two reactive end groups that may incorporate into the parent polymer chain, but may feature multiple arms or reactive ends (e.g. trifunctional and tetrafunctional crosslinkers). Additionally, cross-linkers may have short, discreet molecular structures between reactive end groups, or may have long polymeric spacers between end groups. Table 3 summarizes the features of exemplary cross-linkers used in the examples.

TABLE 3
Summary of covalent cross-linkers utilized in this work.

| Name | Acronym | Functionality | Spacer | Chemical Structure |
|---|---|---|---|---|
| N,N-methylene bisacrylamide | BIS | Bifunctional, 2 | Discreet | 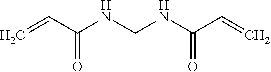 |
| Poly(ethylene glycol) diacrylate Mn = 250 kDA | 250 PEG- DA | Bifunctional, 2 | Polymeric, Mn = 250 | 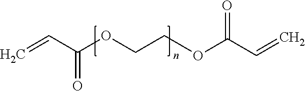 |
| Poly(ethylene glycol) diacrylate Mn = 575 kDA | 575 PEG- DA | Bifunctional, 2 | Polymeric, Mn = 575 | 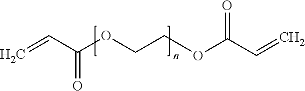 |
| Poly(ethylene glycol) diacrylate, Mn = 700 kDa | 700 PEG- DA | Bifunctional, 2 | Polymeric, Mn = 700 | 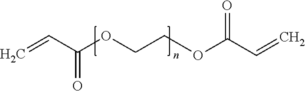 |
| Poly(propylene glycol) diacrylate, Mn = 800 kDa | 800 PPG- DA | Bifunctional, 2 | Polymeric, Mn = 800 | 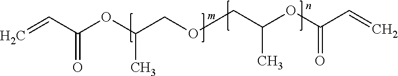 |
| Trimethylolpropane triacrylate | TMP-TA | Trifunctional, 3 | Discreet | 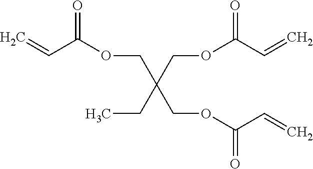 [SMILES: C=CC(=O)OCC(CC)(COC(=O)C=C)COC(=O)C=C] |
| Trimethylolpropane ethoxylate triacrylate, Mn = 428 kDa | 428 TMPE-TA | Trifunctional, 3 | Polymeric, Mn = 428 | 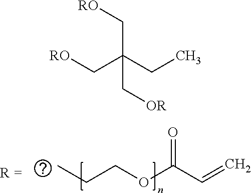 |
| Trimethylolpropane ethoxylate triacrylate, Mn = 912 kDa | 912 TMPE-TA | Trifunctional, 3 | Polymeric, Mn = 912 | 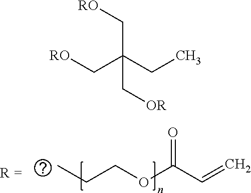 |
| Pentaerythritol tetraacrylate | PE-TA | Tetrafunctional, 4 | Discreet | 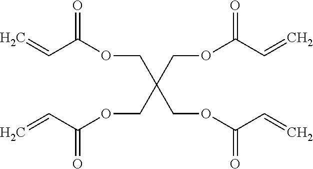 [SMILES: C=CC(=O)OCC(COC(=O)C=C)(COC(=O)C=C)COC(=O)C=C] |
| Dipentaerythritol penta-/hexa- acrylate | DPE-HA | Hexafunctional, 6 | Discreet | 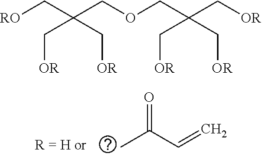 |

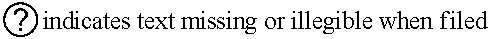

Typically, bi- and trifunctional cross-linking agents may be used to synthesize hydrogel meshes of this disclosure. The use of other polyfunctional cross-linkers for the mesh synthesis resulted in poorly formed meshes that exhibit extreme fragility, and were not physically stable enough for further characterization.

### Example 5. Comonomers and Additives

Comonomers and additives were explored for their role in mesh synthesis and impact on the final mesh product. By definition, comonomers are reagents that may covalently bind and incorporate into the parent chain formed during photo polymerization. Additives may be components that are entrapped in the interpenetrating cross-linked polymer network. Additives may bear functional groups that facilitate intramolecular interactions with the cross-linked polymer network, but they may not be covalently bonded to the polymer network. Table 3 summarizes the additives and comonomers used in this work.

TABLE 3
Exemplary comonomers and additives used in the hydrogel mesh synthesis.

| Name | Class | Features | Structure |
|---|---|---|---|
| 2-ethylhexyl acrylate | Comonomer | Discreet Hydrophobic Comonomer | 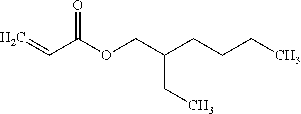 |
| Di(ethylene glycol) 2-ethylhexyl acrylate^{†} | Comonomer | Discreet Hydrophobic Comonomer | 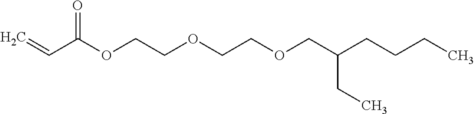 [SMILES: C=CC(=O)OCCOCCOCC(CC)CCCC] |
| Poly(ethylene glycol) methyletheracrylate | Comonomer | Polymeric Comonomer, Mn = 480 | 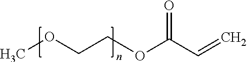 |
| Poly(propylene glycol) acrylate | Comonomer | Polymeric Comonomer, Mn = 475 | 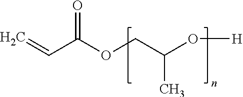 |
| Sodium acrylate | Comonomer | Ionic Comonomer | 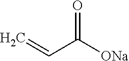 |
| 2-hydroxyethyl acrylate | Comonomer | Discreet Hydrophilic Comonomer | 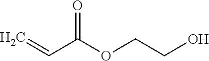 |
| 2- hydroxy- methylethyl acrylate | Comonomer | Discreet Hydrophilic Comonomer | 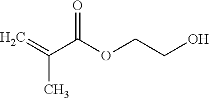 |
| N-(3,4- dihydroxy- phenethyl) acrylamide | Comonomer | Discreet Hydrophilic Comonomer | 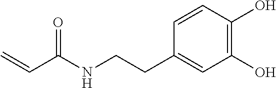 |
| Laponite | Additive | Inorganic clay | |
| Sodium Polyacrylate | Additive | Polymeric cross-linked microbeads | 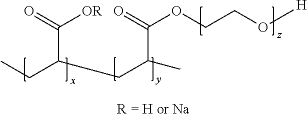 |
| Sodium Alginate | Additive | Polysaccharide | 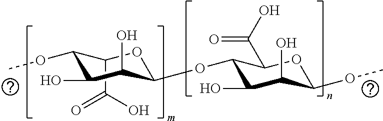 |
| Chitosan | Additive | Polysaccharide | 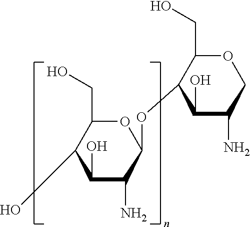 |
| Tannic Acid | Additive | Polyphenol | 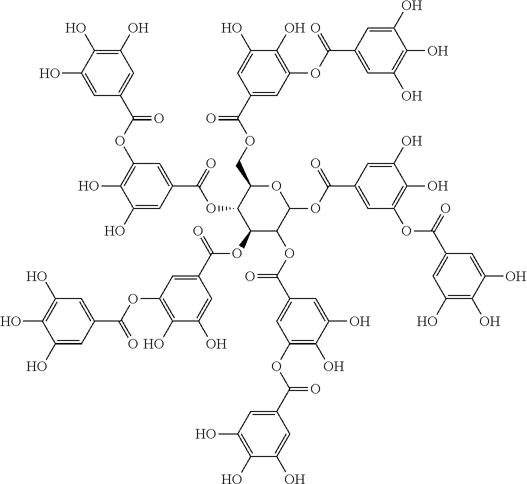 [SMILES: O=C(OC[C@H]1OC(OC(=O)c2cc(O)c(O)c(OC(=O)c3cc(O)c(O)c(O)c3)c2)C(OC(=O)c2cc(O)c(O)c(OC(=O)c3cc(O)c(O)c(O)c3)c2)[C@@H](OC(=O)c2cc(O)c(O)c(OC(=O)c3cc(O)c(O)c(O)c3)c2)[C@@H]1OC(=O)c1cc(O)c(O)c(OC(=O)c2cc(O)c(O)c(O)c2)c1)c1cc(O)c(O)c(OC(=O)c2cc(O)c(O)c(O)c2)c1] |

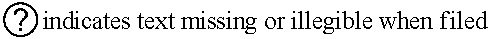

Some polymeric comonomers did not improve adhesive or tensile strength of the hydrogel meshes. Some hydrogel meshes that were synthesize by using hydrophilic comonomers were either weakly adhesive or did not have complete polymerization. Some hydrogel meshes synthesized by using laponite or sodium polyacrylate additives were fragile and significantly swelled in water. In some cases, polysaccharides (alginate and chitosan) had poor solubility in formulation solvents, even at low weight loadings, and did not improve mesh properties.

Some formulations used tannic acid as an additive during a post-polymerization humectant solvent exchange washing process. In most cases, the addition of tannic acid improved adhesive strength at both cold compress and body temperature conditions. These formulations are represented in Table 3B.

TABLE 3B
Exemplary polymer formulations with tannic acid additive

| | NIPAM | AA | Cross-linker | Comonomer | Tannic |
|---|---|---|---|---|---|
| Formu- | (% | (% | (Identity, % | (Identity, | Acid |
| lation | w/v) | w/v) | w/v) | % w/v) | (% w/v) |
| M-181 | 10 | 10 | PEG-DA, 700 | — | 1 |
| | | | Mn, 1 | | |
| M-254 | 10 | 10 | PPG-DA, 800 | EHA, 0.5 | 1 |
| | | | Mn, 1 | | |
| M-254 | 10 | 10 | PPG-DA, 800 | EHA, 0.5 | 10 |
| | | | Mn, 1 | | |
| M-214 | 10 | 10 | TMP-TA, 1 | — | 1 |

Small feed ratios of hydrophobic comonomers, such as 2-ethylhexyl acrylate (EHA) and di(ethylene glycol) 2-ethylhexyl acrylate (DEG-EHA), helped to improve adhesive strength in some formulations. Generally, feed ratios of about 0.5% EHA in formulations with a PPG-DA cross-linker yielded strongly adhesive finished hydrogel meshes. However, higher EHA feed ratios, or EHA paired with other cross-linkers, yielded chalky, non-adhesive meshes after the ethanol-to-water titrated wash technique. Meshes with EHA loadings greater than about 0.5% may alternatively be washed used an ethanol-humectant-water or humectant-water titrated wash procedure to maintain desirable adhesive properties, which may otherwise be lost during an ethanol-to-water titrated wash.

### Example 6. Post Synthetic Humectant Treatment

Synthetic hydrogel meshes may retain moisture under aqueous storage conditions, but if left to equilibrate under ambient conditions (about 50% RH, room temperature for 48 hours), they may dry out and lose their desirable properties. Upon drying, a formerly adhesive, flexible, and elastic mesh may eventually become non-adhesive, hard, and brittle. This may be an issue for final product packaging, delivery, and may limit the hydrogel meshes' length of application. Treating the finished hydrogel meshes with humectants, additives that promote moisture retention, may be one approach to help meshes maintain or enhance desirable properties. Some commercially available wound dressing products most commonly contain glycerol as a humectant. Humectants are commonly found and used in beauty products. Many humectants pose little to no toxicity risk.

Following three exemplary humectants have been screened for their utility in hydrogel meshes: glycerol, propylene glycol, and hexylene glycol (Table 4). Each humectant was screened with formulation M-254 and first evaluated for their post-treatment qualitative properties.

TABLE 4
Exemplary humectants used to wash the hydrogel gels.

| Name | CAS No. | Supplier | SKU or P/N | Structure |
|---|---|---|---|---|
| Glycerol^{†} | 56-81-5 | Sigma-Aldrich | G9012 | 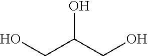 |
| Propylene glycol^{†} | 57-55-6 | Sigma-Aldrich | W294004 | 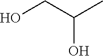 |
| Hexylene glycol^{‡} | 107-41-5 | Sigma-Aldrich | 112100 | 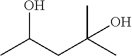 |

### Example 7. Humectant Treatment Procedure

There may be three approaches for treating finished meshes with humectants. First, the mesh can be subjected to the traditional ethanol-to-water titrated wash procedure, followed by a water-to-humectant treatment. Second, the traditional ethanol-to-water titrated wash can be skipped entirely and the mesh can be subjected to an ethanol-humectant-water titrated wash. Third, a synthetic mesh that utilized propylene glycol as a polymerization solvent can be subjected to a humectant-water wash. The first method was utilized for samples M-254 and M-181. The second method was utilized for M-252, M-254, M-272, and M-279. The third method was utilized for samples M-308.

TABLE 5

| Wash | Ethanol | Propylene | Water |
|---|---|---|---|
| No. | (% v/v) | Glycol (% v/v) | (% v/v) |
| 1&2 | 100 | 0 | 0 |
| 3 | 75 | 25 | 0 |
| 4 | 50 | 50 | 0 |
| 5 | 25 | 50 | 25 |
| 6&7 | 0 | 75 | 25 |

The ethanol-humectant-water titrated wash procedure has the following steps (Summarized in Table 5):
(1) Submerge in ethanol, 200 proof, and rock for about 15 minutes. Decant off liquid.
(2) Submerge in ethanol, 200 proof, and rock for about 15 minutes (again). Decant off liquid.
(3) Submerge in ethanol/propylene glycol, 75/25 v/v, and rock for about 15 minutes. Decant off liquid.
(4) Submerge in ethanol/propylene glycol, 50/50 v/v, and rock for about 15 minutes. Decant off liquid.
(5) Submerge in ethanol/propylene glycol/water, 25/50/25 v/v, and rock for about 15 minutes. Decant off liquid.
(6) Submerge in propylene glycol/water, 75/25 v/v, and rock for about 15 minutes. Decant off liquid.
(7) Submerge in propylene glycol/water, 75/25 v/v, and rock for about 15 minutes (again). Decant off liquid.

The humectant-water wash procedure has the following steps:

(1) Submerge in propylene glycol/water solution, and rock for 15 minutes. The glycol/water ratio may be in a range of 50/50 v/v to 75/25 v/v. Then, decant off the liquid.

(2) Submerge in propylene glycol/water solution, and rock for 15 minutes. The glycol/water ratio may be in a range of 50/50 v/v to 75/25 v/v. Decant off the liquid.

After completion of the wash procedure, the hydrogel meshes are air dried on the non-adhesive side of 3M release liner. The final hydrogel mesh is clear, colorless, and very tacky to touch.

### Example 8. Exemplary Reversible Adhesive Hydrogel Mesh Properties

Plus and minus symbols are used to designate if the humectant treated mesh has the characteristic (+) or not (−), and if the characteristic appears to be more prominent compared to others (+, ++, or +++).

TABLE 6
Characteristic after about 24 hours

| | | | | | | | | Adhesive | | | |
|---|---|---|---|---|---|---|---|---|---|---|---|
| | | Clear | | | | | | with | Rigid | Rigid | |
| Sample | Assignment | Texture | Colorless | Flat | Flexible | Elastic | Tacky | Pressure | Edges | Center | Deformation |
| 1B | PPG | + | + | + | + | + | + | ++ | + | − | − |
| | 25% | | | | | | | | | | |
| 1C | PPG | + | + | + | ++ | +++ | +++ | +++ | − | − | − |
| | 50% | | | | | | | | | | |
| 1D | PPG | + | + | − | + | + | − | + | − | + | + |
| | 75% | | | | | | | | | | |
| 3C | PPG | − | + | − | − | − | + | + | − | ++ | ++ |
| | 100% | | | | | | | | | | |
| 2B | HG 25% | + | + | + | + | − | − | + | + | − | − |
| 2C | HG 50% | + | + | + | +++ | +++ | +++ | +++ | − | − | − |
| 2D | HG 75% | + | + | + | +++ | +++ | +++ | +++ | − | − | − |
| 3D | HG | − | + | − | + | − | − | ++ | − | − | + |
| | 100% | | | | | | | | | | |

TABLE 7
Characteristic after 5 days

| | | | | | | | Adhesive | | | |
|---|---|---|---|---|---|---|---|---|---|---|
| | | Clear | | | | | with | Rigid | Rigid | |
| Sample | Assignment | Texture | Colorless | Flat Flexible | Elastic | Tacky | Pressure | Edges | Center | Deformation |
| 1B | PPG 25% | + | + | + | − | + | + | + | ++ | − |
| 1C | PPG 50% | + | + | + | − | +++ | + | + | + | − |
| 1D | PPG 75% | + | + | ++ | + | ++ | +++ | − | − | − |
| 3C | PPG 100% | + | + | ++ | − | ++ | +++ | − | − | − |
| 2B | HG 25% | + | + | + | − | − | − | + | ++ | − |
| 2C | HG 50% | + | + | +++ | ++ | +++ | +++ | + | − | − |
| 2D | HG 75% | + | + | ++++ | +++ | +++ | +++ | − | − | − |
| 3D | HG 100% | + | + | ++ | − | ++ | ++ | − | − | − |

TABLE 8
Characteristic after 15 days

| | | | | | | | | Adhesive | | | |
|---|---|---|---|---|---|---|---|---|---|---|---|
| | | Clear | | | | | | with | Rigid | Rigid | |
| Sample | Assignment | Texture | Colorless | Flat | Flexible | Elastic | Tacky | Pressure | Edges | Center | Deformation |
| 1B | PPG 25% | + | + | + | − | − | + | + | + | ++ | − |
| 1C | PPG 50% | + | + | + | − | − | + | + | + | − | − |
| 1D | PPG 75% | + | + | + | +++ | +++ | +++ | +++ | − | − | − |
| 3C | PPG 100% | + | + | + | +++ | +++ | +++ | +++ | − | − | − |
| 2B | HG 25% | + | + | + | − | − | − | − | + | ++ | − |
| 2C | HG 50% | + | + | + | + | + | ++ | ++ | + | | − |
| 2D | HG 75% | + | + | + | +++ | +++ | +++ | +++ | | − | |
| 3D | HG 100% | + | + | + | +++ | +++ | +++ | +++ | | − | − |

TABLE 9

| | | Mass | Mass | Mass | Mass |
|---|---|---|---|---|---|
| | | before | after | after | after |
| | Assign- | Washes | Washes | 5 days | 15 days |
| Sample | ment | (g) | (g) | (g) | (g) |
| 1B | PPG 25% | 2.1318 | 1.9884 | 1.7875 | 1.6704 |
| 1C | PPG 50% | 1.8450 | 1.8981 | 1.6403 | 1.5865 |
| 1D | PPG 75% | 1.9051 | 2.6331 | 2.4534 | 2.4966 |
| 3C | PPG 100% | 1.4575 | 2.3640 | 2.3682 | 2.4662 |
| 2B | HG 25% | 1.8900 | 1.9271 | 1.3551 | 1.3288 |
| 2C | HG 50% | 1.9497 | 2.4963 | 1.9475 | 1.8634 |
| 2D | HG 75% | 1.9032 | 2.6421 | 2.1150 | 1.9861 |
| 3D | HG 100% | 1.5848 | 3.4120 | 3.1765 | 3.2049 |

TABLE 10
Characteristic after 1 day

| | | | | | | | | Adhesive | | | |
|---|---|---|---|---|---|---|---|---|---|---|---|
| | | Clear | | | | | | with | Rigid | Rigid | |
| Sample | Assignment | Texture | Colorless | Flat | Flexible | Elastic | Tacky | Pressure | Edges | Center | Deformation |
| M-252 | PPG 75% | + | + | + | +++ | − | ++ | ++ | − | − | − |
| M-254 | PPG 75% | + | + | + | +++ | − | + | + | − | − | − |
| M-273 | PPG 75% | + | + | + | +++ | − | +++ | +++ | − | − | − |

TABLE 11
Characteristic after 10 days

| | | | | | | | | Adhesive | | | |
|---|---|---|---|---|---|---|---|---|---|---|---|
| | | Clear | | | | | | with | Rigid | Rigid | |
| Sample | Assignment | Texture | Colorless | Flat | Flexible | Elastic | Tacky | Pressure | Edges | Center | Deformation |
| M-252 | PPG 75% | + | + | + | +++ | + | ++ | ++ | − | − | − |
| M-254 | PPG 75% | + | + | + | +++ | + | + | + | − | − | − |
| M-273 | PPG 75% | + | + | + | +++ | + | +++ | +++ | − | − | − |

### Example 9. Rheology for Thermal Behavior

The lower critical solution temperature (LCST) of prepared meshes was measured by rheology on a TA Instruments Discovery Hybrid Rheometer using a 20 mm Peltier Plate with temperature control and immersion cup attachment. All samples were measured while submerged in distilled water. All samples were measured while using active axial force control in compression mode set to maintain about 0.25 N of applied force with about 0.1 N sensitivity. Circular samples of the meshes, about 20 mm in diameter, were prepared using a die punch. Prior to measurement, samples were submerged and equilibrated in the immersion cup at about 10° C. for about 5 minutes before securing between the Peltier plate. First, the viscoelastic region of the meshes was determined by performing a strain sweep on a fresh sample at five different temperatures (10° C., 25° C., 30° C., 35° C., 40° C.). A single strain value, which falls in the viscoelastic region of the mesh at each temperature, was selected for each sample and used for subsequent rheology measurements. Second, a fresh sample was measured at the predetermined strain setting under an oscillation temperature sweep, cycled between 10° C. and 40° C. The experiment was programmed to let the sample equilibrate to each temperature point for about 5 minutes prior to application of strain. The LCST for each sample was identified by the inflection point in which the measured GAP starts decreasing as the sample is heated.

TABLE 12
LCST of select meshes, as measured by rheometry.

| Formu- | NIPAM | Cross-linker | AA | Other | LCST |
|---|---|---|---|---|---|
| lation | (w/v %) | (w/v %) | (w/v %) | (w/v %) | (° C.) |
| M-179 | 10 | 1, 700 PEG-DA | 15 | — | 25 |
| M-181 | 10 | 1, 700 PEG-DA | 10 | — | 26 |
| M-182 | 10 | 1, 800 PPG-DA | 10 | — | 32 |
| M-183 | 10 | 1, BIS | 10 | — | 30 |
| M-184 | 10 | 1, 575 PEG-DA | 10 | — | 27 |
| M-185 | 10 | 1, 250 PEG-DA | 10 | — | 27 |
| M-192 | 10 | 1, 700 PEG-DA | — | — | 27 |
| M-195 | 10 | 1, 700 PEG-DA | 5 | — | 26* |
| M-212 | 15 | 1, 700 PEG-DA | 10 | — | 27 |
| M-213 | 10 | 1, 428 TMPE-TA | 10 | — | 25 |
| M-214 | 10 | 1, TMP-TA | 10 | — | 25 |
| M-216 | 10 | 1, 912 TMPE-TA | 10 | — | 24 |
| M-220 | 10 | 1, 428 TMPE-TA | 10 | 0.5, SPA | 23 |
| M-221 | 10 | 1, TMP-TA | 10 | 0.5, SPA | 26 |
| M-222 | 10 | 1, 912 TMPE-TA | 10 | 0.5, SPA | |
| M-233 | 10 | 1, 700 PEG-DA | 10 | 1, PEG-MEA | 25 |
| M-234 | 10 | 1, 700 PEG-DA | 10 | 5, PEG-MEA | 26 |
| M-235 | 10 | 1, 700 PEG-DA | 5 | — | |
| M-236 | 10 | 1, 800 PPG-DA | 10 | 1, PPG-A | 25 |
| M-237 | 10 | 1, 800 PPG-DA | 10 | 5, PPG-A | 17 |
| M-240 | 10 | 1, 700 PEG-DA | 10 | 1, PPG-A | 22 |
| M-241 | 10 | 1, 700 PEG-DA | 10 | 5, PPG-A | |
| M-245 | 10 | 2, 575 PEG-DA | — | — | 34 |
| M-246 | 10 | 2, 250 PEG-DA | — | — | |
| M-247 | 10 | 2, BIS | — | — | |
| M-254 | 10 | 1, 800 PPG-DA | 10 | 0.5, EHA | 25 |
| M-276 | 5 | 1, 700 PEG-DA | 10 | — | N.D. |

N.D. means “not detected.”
“F” means the mesh was too fragile to analyze by rheometry method.
TBA—we have the raw data file, need to complete analysis.

### Example 10. Adhesive Strength of Mesh Measured by Tension; ASTM F 2258-05

The adhesive strength of prepared hydrogel meshes was characterized using ASTM method F 2258-05. Synthetic skin derived from porcine gelatin was prepared in house, according to an internal SOP (SOP 2018.001), based on the procedure reported by Lir and coworkers (J. Adhesion Sci. Technol. 2007, 21 (15), 1497-1512. Commercial comparators include tegaderm, a pressure sensitive medical adhesive, and various hydrogel adhesives.

Tension measurements are completed under two primary temperature conditions as a means to screen the materials: cold compress (about 15° C.) and body temperature (about 37° C.). In many cases, there are no significant differences in measured tension of a hydrogel mesh to synthetic skin at the two different temperature conditions, even though there is a detected LCST of the mesh in rheology experiments. Hydrogel meshes that do not exhibit thermal triggers under adhesion tests often still have other external environmental triggers that can be utilized to turn off adhesion. Namely, application of aqueous solutions with specific ionic strengths, ionic compositions, or pH may cause a release in adhesion. The tension tests may also be a valuable measurement technique, because when combined as a whole with other experiments and qualitative observations, we may observe which formulations may be ruled out as a promising material. In some cases, hydrogel meshes may be prepared and appear as a mesh with good internal mechanical strength (to prevent cohesive failure, or tearing during a test), but testing reveals the hydrogel mesh may be too week to mechanically characterize for adhesive strength.

### Example 11. Results from Tension Tests

Table 13 summarizes tension data of adhesive hydrogel meshes on synthetic skin collected under different temperature conditions. NC indicates “not characterizable,” because trials resulted in significant cohesive failure of the mesh. Non characterizable meshes are generally tear easily or are brittle. Data is reported as the tension value±the standard deviation.

TABLE 13

| | | Cold | Body |
|---|---|---|---|
| | | Compress, | Temperature, |
| Formu- | | 15° C. | 37° C. |
| lation | Treatment | (N/cm²) | (N/cm²) |
| Tegaderm | — | 0.48 ± 0.07 | 0.81 ± 0.11 |
| M-181 | Water-Borne | 0.48 ± 0.17 | 0.46 ± 0.04 |
| M-181 | Humectant-Borne, | 1.11 ± 0.48 | 0.99 ± 0.52 |
| | 75:25 | | |
| | PPG:Water with 1% | | |
| | w/v Tannic Acid | | |
| M-182 | Water-Borne | 0.76 ± 0.04 | 0.82 ± 0.16 |
| M-183 | Water-Borne | 0.51 ± 0.11 | 0.59 ± 0.22 |
| M-184 | Water-Borne | 0.64 ± 0.10 | 0.58 ± 0.11 |
| M-185 | Water-Borne | 0.90 ± 0.30 | 1.24 ± 0.47 |
| M-204 | Water-Borne | NC | NC |
| M-211 | Water-Borne | 0.68 ± 0.24 | 0.84 ± 0.32 |
| M-212 | Water-Borne | 0.62 ± 0.18 | 0.71 ± 0.22 |
| M-213 | Water-Borne | 0.93 ± 0.55 | 0.99 ± 0.21 |
| M-214 | Water-Borne | 0.53 ± 0.09 | 1.32 ± 0.22 |
| M-214 | Humectant-Borne, | 0.33 ± 0.22 | 0.37 ± 0.22 |
| | 75:25 PPG:Water | | |
| M-214 | Humectant-Borne, | 0.31 ± 0.12 | 0.38 ± 0.20 |
| | 75:25 | | |
| | PPG:Water with 1% | | |
| | w/v Tannic Acid | | |
| M-216 | Water-Borne | 0.69 ± 0.29 | 0.88 ± 0.28 |
| M-233 | Water-Borne | 0.69 ± 0.27 | 0.44 ± 0.0 |
| M-234 | Water-Borne | 0.56 ± 0.30 | 0.72 ± 0.19 |
| M-254 | Water-Borne | 1.22 ± 0.14 | 1.33 ± 0.38 |
| M-236 | Water-Borne | 1.89 ± 0.70 | 1.51 ± 0.10 |
| M-237 | Water-Borne | 2.34 ± 0.81 | 1.91 ± 0.36 |
| M-254 | Humectant-Borne, | 0.51 ± 0.16 | 0.61 ± 0.06 |
| | 75:25 PPG:Water | | |
| M-254 | Humectant-Borne, | 2.18 ± 0.58 | 2.70 ± 1.12 |
| | 75:25 | | |
| | PPG:Water with 1% | | |
| | w/v Tannic Acid | | |
| M-254 | Humectant-Borne, | 1.79 ± 1.02 | 1.46 ± 1.1 |
| | 75:25 | | |
| | PPG:Water with 10% | | |
| | w/v Tannic Acid | | |
| M-276 | Water-Borne | 1.02 ± 0.25 | NC |
| M-278 | Humectant-Borne, | 0.79 ± 0.39 | 0.88 ± 0.30 |
| | 75:25 PPG:Water | | |
| M-279 | Humectant-Borne, | 0.85 ± 0.22 | 2.13 ± 0.81 |
| | 75:25 PPG:Water | | |
| M-280 | Water-Borne | 1.38 ± 0.07 | 1.31 ± 0.20 |
| M-281 | Water-Borne | 0.38 ± 0.14 | 0.30 ± 0.11 |
| M-282 | Water-Borne | 1.20 ± 0.50 | 0.98 ± 0.25 |
| M-283 | Water-Borne | 0.70 ± 0.09 | 0.52 ± 0.24 |
| M-284 | Water-Borne | 1.07 ± 0.34 | 0.70 ± 0.22 |
| M-285 | Water-Borne | 1.35 ± 0.07 | 1.50 ± 0.00* |
| M-286 | Water-Borne | 2.11 ± 0.35 | 1.77 ± 0.19 |
| M-287 | Water-Borne | 0.72 ± 0.09 | 1.44 ± 0.72 |
| M-288 | Water-Borne | 2.04 ± 0.29 | 0.52 ± 0.25 |
| M-289 | Water-Borne | 2.33 ± 0.40 | 2.93 ± 0.57 |
| M-290 | Water-Borne | 2.70 ± 0.36 | 1.39 ± 0.28 |
| M-293 | Water-Borne | 1.24 ± 0.39 | 0.81 ± 0.12 |
| M-294 | Water-Borne | 0.77 ± 0.17 | 1.17 ± 0.28 |
| M-295 | Water-Borne | 2.42 ± 0.73 | 1.35 ± 0.28 |
| M-296 | Water-Borne | 1.29 ± 0.07 | 1.06 ± 0.17 |
| M-297 | Water-Borne | 0.59 ± 0.06 | 1.19 ± 0.70 |
| M-298 | Water-Borne | 1.43 ± 0.57 | 1.49 ± 0.72 |
| M-299 | Water-Borne | 1.65 ± 0.64 | 0.98 ± 0.21 |
| M-300 | Water-Borne | 1.24 ± 0.42 | 1.00 ± 0.55 |
| M-301 | Water-Borne | 1.86 ± 0.93 | 1.66 ± 0.96 |
| M-302 | Water-Borne | 1.08 ± 0.45 | 1.00 ± 0.35 |

### Example 12. Synthesis/Mesh Formulations/Solvent Treatments

Many formulations have been prepared, but not characterized mechanically, since they were too fragile to be handled. As a general rule, this is true for any hydrogel mesh formulation that does not include both NIPAM and acrylic acid.

Furthermore, if NIPAM and acrylic acid are significantly varied in their composition (meaning NIPAM greatly outweighs AA, or vice versa), then the hydrogel meshes may be too fragile. This may be because the side groups of NIPAM and AA may form intramolecular hydrogen bonds with each other, which may contribute to the cohesive strength of the finished mesh.

Hydrogel meshes with EHA may generally be more adhesive than those without EHA, even at low loadings. However, hydrogel meshes that are “water borne,” may have a limit to how much EHA may be added. If a hydrogel mesh is subjected to the ethanol-to-water titrated wash, the EHA loading may not be more than about 0.5% w/v %. If the loading is greater than about 0.5%, the finished mesh is opaque, feels chalky, and is non-adhesive. No thermal trigger is observable in these cases.

Hydrogel meshes that are treated with humectants may contain greater loadings of EHA and maintain their adhesive characteristics.

EHA may be compatible with PPG-DA cross-linkers. Comparatively, when EHA is combined with PEG-DA cross-linkers, even at low loadings, the overall hydrogel mesh properties may be not suitable. The finished hydrogel meshes feel chalky and non-adhesive.

Humectant treated meshes feel very tacky, meaning they have a strong initial sticking power to substrates. In some cases, humectant treated meshes have weaker overall adhesion compared to their water-borne analogs.

### Example 13. Hydrogel Mesh Behavior During “Curing”

Water borne hydrogel meshes often have a visible change in opacity when it cures to a substrate at body temperature. Under room temperature conditions, the hydrogel meshes are usually clear and colorless. At body temperature conditions, the hydrogel meshes become opaque-white as they adhere to a substrate.

If the opposite side of a hydrogel mesh is exposed to air while its substrate side is curing, the air interface side may become non adhesive after about 5 minutes or longer. This is true for any hydrogel mesh containing EHA, and for some hydrogel meshes without EHA.

All articles, patents, patent applications, and other publications that have been cited in this disclosure are incorporated herein by reference.

In this disclosure, the indefinite article “a” and phrases “one or more” and “at least one” are synonymous and mean “at least one”.

Relational terms such as “first” and “second” and the like may be used solely to distinguish one entity or action from another, without necessarily requiring or implying any actual relationship or order between them. The terms “comprises,” “comprising,” and any other variation thereof when used in connection with a list of elements in the specification or claims are intended to indicate that the list is not exclusive and that other elements may be included. Similarly, an element preceded by an “a” or an “an” does not, without further constraints, preclude the existence of additional elements of the identical type.

The abstract is provided to help the reader quickly ascertain the nature of the technical disclosure. It is submitted with the understanding that it will not be used to interpret or limit the scope or meaning of the claims. In addition, various features in the foregoing detailed description are grouped together in various examples to streamline the disclosure. This method of disclosure should not be interpreted as requiring claimed examples to require more features than are expressly recited in each claim. Rather, as the following claims reflect, inventive subject matter lies in less than all features of a single disclosed example. Thus, the following claims are hereby incorporated into the detailed description, with each claim standing on its own as separately claimed subject matter.

1. A reversible adhesive hydrogel mesh, comprising cross-linked components of the following monomers:
a reversible monomer of a reversible adhesive polymer,
acrylic acid (AA), and
an acrylate cross-linker.

2. The reversible adhesive hydrogel mesh of claim 1, wherein the reversible monomer of the reversible adhesive polymer comprises N-methyl-N-n-propylacrylamide, N-n-propylacrylamide, N-methyl-N-isopropylacrylamide, N-n-propylmethacrylamide, N-isopropylacrylamide (NIPAM), N-n-diethylacrylamide, N-isopropylmethacrylamide, N-cyclopropylacrylamide, N-ethylmethyacrylamide, N-methyl-N-ethylacrylamide, N-cyclopropylmethacrylamide, N-ethylacrylamide, N-n-diethylacrylamide, or a mixture thereof.

3. The reversible adhesive hydrogel mesh of claim 1, wherein the acrylate cross-linker comprises a multifunctional acrylate cross-linker.

4. The reversible adhesive hydrogel mesh of claim 1, wherein the acrylate cross-linker comprises a bifunctional acrylate cross-linker, a trifunctional acrylate cross-linker, a tetrafunctional acrylate cross-linker, a hexafunctional acrylate cross-linker, or a mixture thereof.

5. The reversible adhesive hydrogel mesh of claim 1, wherein the acrylate cross-linker comprises a bifunctional acrylate cross-linker, a trifunctional acrylate cross-linker, or a mixture thereof.

6. The reversible adhesive hydrogel mesh of claim 1, wherein the acrylate cross-linker comprises N,N-methylene bisacrylamide (BIS), a poly(ethylene glycol) diacrylate (PEG-DA), trimethylolpropane triacrylate (TMP-TA), a trimethylolpropane ethoxylate triacrylate, (TMPE-TA), poly(propylene glycol) diacrylate (PPG-DA), trimethylolpropane triacrylate, or a mixture thereof.

7. The reversible adhesive hydrogel mesh of claim 1, wherein the acrylate cross-linker comprises a covalent diacrylate cross-linker, and wherein the covalent diacrylate cross-linker comprises N,N-methylene bisacrylamide (BIS), poly(ethylene glycol) diacrylate (PEG-DA), poly(propylene glycol) diacrylate (PPG-DA), or a mixture thereof.

8. The reversible adhesive hydrogel mesh of claim 7, wherein the acrylate cross-linker comprises PEG-DA with an average molecular weight, Mn of about 250 kDa; PEG-DA with an average molecular weight, Mn of about 575 kDa; PEG-DA with an average molecular weight, Mn of about 700 kDa; PPG-DA with an average molecular weight Mn of about 800 kDa; TMPE-TA with an average molecular weight, Mn of about 428 kDa; TMPE-TA with an average molecular weight, Mn of about 912 kDa; or a mixture thereof.

9-73. (canceled)

74. A method of preparation of a reversible adhesive hydrogel mesh, comprising:
preparing a reaction solution comprising a polymer formulation, wherein the polymer formulation comprises:
a reversible monomer of a reversible adhesive polymer,
acrylic acid (AA),
an acrylate cross-linker,
a catalyst, and
a solvent,
reacting the polymer formulation to prepare the reversible adhesive hydrogel mesh, and
treating the prepared reversible adhesive hydrogel mesh with a treatment solution comprising a humectant, and thereby
obtaining a reversible adhesive hydrogel mesh with improved tack.

75. (canceled)

76. The method of preparation of a reversible adhesive hydrogel mesh of claim 74, wherein the treating the prepared reversible adhesive hydrogel mesh comprises: treating the reversible adhesive hydrogel mesh with a first treatment solution before treating the prepared reversible adhesive hydrogel mesh with a treatment solution comprising a humectant, wherein the first treatment solution comprises water and an alcohol; and then treating the reversible adhesive hydrogel mesh with a second treatment solution, wherein the second treatment solution comprises a humectant and water.

77. The method of preparation of a reversible adhesive hydrogel mesh of claim 74, wherein the treating the prepared reversible adhesive hydrogel mesh comprises: treating the reversible adhesive hydrogel mesh with a treatment solution, wherein the treatment solution comprises a humectant, an alcohol and water; and thereby obtaining a reversible adhesive hydrogel mesh with improved tack.

78. The method of preparation of a reversible adhesive hydrogel mesh of claim 74, wherein the treating the prepared reversible adhesive hydrogel mesh comprises: treating the reversible adhesive hydrogel mesh with a treatment solution, wherein the treatment solution comprises a humectant and water; and thereby obtaining a reversible adhesive hydrogel mesh with improved tack.

79. The method of preparation of a reversible adhesive hydrogel mesh of claim 74, wherein the humectant comprises glycerol, ethylene glycol, propylene glycol, hexylene glycol, an aloe extract, hyaluronic acid, 2,3-butanediol, butyl ethyl propanediol, or a mixture thereof.

80. The method of preparation of a reversible adhesive hydrogel mesh of claim 74, wherein the humectant concentration of a treatment solution is equal to or higher than 25 volume percent, equal to or higher than 50 volume percent, or equal to or higher than 75 volume percent in the mixture comprising a humectant and water.

81. (canceled)

82. The method of claim 74, wherein the polymer formulation further comprises an acrylate co-monomer.

83. The method of claim 74, wherein the solvent comprises dimethylformamide (DMF), water, ethanol, ethyl acetate, propylene carbonate, dimethyl sulfoxide (DMSO), propylene glycol, or a mixture thereof.

84. The method of claim 74, wherein the catalyst comprises a photo-initiator for free radical polymerization.

85. The method of claim 74, wherein the catalyst comprises potassium persulfate, azobisisobutyronitrile, 4,4′-azobis(4-cyanovaleric acid), 1,1′-azobis(cyclohexanecarbonitrile), 2-hydroxy-4′-(2-hydroxyethoxy)-2-methylpropiophenone, or a mixture thereof.

86. The method of claim 74, wherein the polymer formulation further comprises laponite, sodium polyacrylate, sodium alginate, tannic acid, chitosan, a silver particle, an aloe extract, propanediol, 1,4-butanediol, 1,5-pentanediol, hexanediol, octanediol, or a mixture thereof.

87. The method of claim 74, wherein the polymer formulation further comprises an acrylate co-monomer; wherein the acrylate co-monomer comprises 2-ethylhexyl acrylate, di(ethylene glycol) 2-ethylhexyl acrylate, poly(ethylene glycol) methyletheracrylate (PEG-MEA), poly(propylene glycol) acrylate (PPG-A), sodium acrylate, 2-hydroxyethyl acrylate, 2-hydroxymethylethyl acrylate, dopamine acrylate, or a mixture thereof.

88. The method of claim 74, wherein the polymer formulation further comprises a vinyl functionalized co-monomer.

89. The method of claim 74, wherein the polymer formulation further comprises vinyl acetate, 1-vinyl-2-pyrrolidinone, N-vinylcaprolactam, ethyl vinyl ether, isobutyl vinyl ether, propyl vinyl ether, butyl vinyl ether, cyclohexyl vinyl ether, tert-butyl vinyl ether, dodecylvinyl ether, 2-ethylhexyl vinyl ether, isopropyl vinyl ether, isobutyl vinyl ether, octyl vinyl ether, ethylene glycol vinyl ether, diethyl vinyl orthoformate, di(ethylene glycol) vinyl ether, 1,4-butanediol vinyl ether, 2-chloroethyl vinyl ether, or a mixture thereof.

90. The method of claim 74, wherein a concentration of the reversible monomer in the polymer formulation is in a range of 1 w/v % to 20 w/v %, wherein a concentration of AA in the polymer formulation is in a range of 1 w/v % to 20 w/v %, wherein a concentration of the acrylate cross-linker in the polymer formulation is in a range of 0.1 w/v % to 5 w/v %, and wherein a concentration of the catalyst is in a range of 0.001 w/v % to 0.5 w/v %.

91. The reversible adhesive hydrogel mesh of claim 1, wherein:
the reversible adhesive hydrogel mesh has an adhesive strength;
the adhesive strength of the reversible adhesive hydrogel mesh at a first temperature is higher than that of the reversible adhesive hydrogel mesh at a second temperature; and
the first temperature is higher than the second temperature.

92. The method of claim 74, wherein:
the reversible adhesive hydrogel mesh has an adhesive strength;
the adhesive strength of the reversible adhesive hydrogel mesh at a first temperature is higher than that of the reversible adhesive hydrogel mesh at a second temperature; and
the first temperature is higher than the second temperature.

93. The reversible adhesive hydrogel mesh of claim 1, wherein:
the reversible adhesive hydrogel mesh has an adhesive strength;
the adhesive strength of the reversible adhesive hydrogel mesh at a first temperature is higher than that of the reversible adhesive hydrogel mesh at a second temperature;
the first temperature is higher than the second temperature;
the adhesive strength of the reversible adhesive hydrogel mesh at the first temperature is in a range of 0.70 N/cm² to 2.20 N/cm², or 0.70 N/cm² to 0.90 N/cm²;
the adhesive strength of the reversible adhesive hydrogel mesh at the second temperature is in a range of 0.40 N/cm² to 0.90 N/cm², or 0.50 N/cm² to 0.80 N/cm², or 0.53 N/cm² to 0.70 N/cm²; and
the adhesive strength of the reversible adhesive hydrogel mesh is characterized using ASTM method F2258-05.

94. The method of claim 74, wherein:
the reversible adhesive hydrogel mesh has an adhesive strength;
the adhesive strength of the reversible adhesive hydrogel mesh at a first temperature is higher than that of the reversible adhesive hydrogel mesh at a second temperature;
the first temperature is higher than the second temperature;
the adhesive strength of the reversible adhesive hydrogel mesh at the first temperature is in a range of 0.70 N/cm² to 2.20 N/cm², or 0.70 N/cm² to 0.90 N/cm²;
the adhesive strength of the reversible adhesive hydrogel mesh at the second temperature is in a range of 0.40 N/cm² to 0.90 N/cm², or 0.50 N/cm² to 0.80 N/cm², or 0.53 N/cm² to 0.70 N/cm²; and
the adhesive strength of the reversible adhesive hydrogel mesh is characterized using ASTM method F2258-05.

95. The method of claim 74, wherein the acrylate cross-linker comprises PEG-DA with an average molecular weight, Mn of about 250 kDa; PEG-DA with an average molecular weight, Mn of about 575 kDa; PEG-DA with an average molecular weight, Mn of about 700 kDa; PPG-DA with an average molecular weight Mn of about 800 kDa; TMPE-TA with an average molecular weight, Mn of about 428 kDa; TMPE-TA with an average molecular weight, Mn of about 912 kDa; or a mixture thereof.
